(19) United States
(12) Patent Application Publication
(10) Pub. No.: US 2022/0226466 A1
(43) Pub. Date: Jul. 21, 2022

(54) PHARMACEUTICAL AGENT FOR INDUCING SPECIFIC IMMUNITY AGAINST SARS-COV-2

(71) Applicant: Federal State Budgetary Institution 'National Research Centre For Epidemiology And Microbiology Name, Moscow (RU)

(72) Inventors: Olga Vadimovna ZUBKOVA, Khimki (RU); Tatiana Andreevna OZHAROVSKAIA, Kotolev (RU); Inna Vadimovna DOLZHIKOVA, Lobnya (RU); Olga POPOVA, Ufa (RU); Dmitrii Viktorovich SHCHEBLIAKOV, Moscow (RU); Daria Mikhailovna GROUSOVA, Moscow (RU); Alina Shahmirovna DZHARULLAEVA, Moscow (RU); Amir Ildarovich TUKHAVTULIN, Moscow (RU); Natalia Mikhailovna TUKHVATULINA, Moscow (RU); Dmitrii Nikolaevich SHCHERBININ, Moskovskaya oblast (RU); Ilias Bulatovich ESMAGAMBETOV, Dmitrov (RU); Elizaveta Alexsandrovna TOKARSKAYA, Moscow (RU); Andrei Gennadevich BOTIKOV, Moscow (RU); Alina Sergeevna EROXOVA, Bryansk (RU); Fatima Magometovna IZHAEVA, Karahaevo-Cherkesskaya Respublika (RU); Aleksandr Sergeevich SEMIKHIN, Moscow (RU); Sergey Vladimirovich BORISEVICH, Sergiev Posad (RU); Boris Savelievich NARODITSKY, Moscow (RU); Denis Yuryevich LOGUNOV, Moscow (RU); Aleksandr Leonidovich GINTSBURG, Moscow (RU)

(21) Appl. No.: 17/715,741

(22) Filed: Apr. 7, 2022

Related U.S. Application Data
(63) Continuation of application No. PCT/RU2020/000591, filed on Nov. 9, 2020.

(30) Foreign Application Priority Data
Aug. 22, 2020 (RU) 2020127980

(51) Int. Cl. A61K 39/215; A61P 31/14; C12N 15/86; C07K 14/005; A61K 9/19; A61K 47/02; A61K 47/26; A61K 47/10; A61K 47/18

(52) U.S. Cl. CPC A61K 39/215; A61P 31/14; C12N 15/86; C07K 14/005; A61K 9/19; A61K 47/02; C12N 2710/10371; A61K 47/10; A61K 47/183; C12N 2770/20022; C12N 2770/20034; C12N 2710/10343; A61K 47/26

(57) ABSTRACT

The invention relates to biotechnology. The claimed agent can be used for the prevention of SARS-CoV-2.

A pharmaceutical agent may contain component (1), and contains a recombinant human adenovirus serotype genome (26), with an expression cassette selected from SEQ ID NO: 1, SEQ ID NO: 2, or SEQ ID NO: 3, and also contains component (2), comprising an agent selected from (i) a recombinant human adenovirus serotype genome (5), with an expression cassette selected from SEQ ID NO: 1, SEQ ID NO: 2, or SEQ ID NO: 3; or (ii) a recombinant simian adenovirus serotype genome (25), with an expression cassette selected from SEQ ID NO: 4, SEQ ID NO: 2, or SEQ ID NO: 3.

Furthermore, a pharmaceutical agent may contain component (1), comprising an agent comprising a recombinant simian adenovirus serotype genome (25), with an expression cassette selected from SEQ ID NO: 4, SEQ ID NO: 2, or SEQ ID NO: 3, and also contains component (2), comprising an agent comprising a recombinant human adenovirus serotype genome (5), with an expression cassette selected from SEQ ID NO: 1, SEQ ID NO: 2, or SEQ ID NO: 3.

### CROSS-REFERENCE TO RELATED APPLICATIONS

The present application is a continuation application of International Application No. PCT/RU2020/000591, filed Nov. 9, 2020, which claims priority to Russian Patent Application No. 2020127980, filed on Aug. 22, 2020, the contents of both applications are hereby incorporated by reference in their entirety.

### INCORPORATION BY REFERENCE—SEQUENCE LISTING

This application includes an electronically submitted sequence listing in .txt format. The .txt file contains a sequence listing entitled “110620_00314 SequenceListing.txt” which was created on Apr. 6, 2022 and is 163,698 bytes in size. The sequence listing contained in this .txt file is part of the specification and is hereby incorporated by reference herein in its entirety.

### FIELD OF THE INVENTION

The invention relates to biotechnology, immunology and virology. The claimed agent can be used for the prevention of diseases caused by severe acute respiratory syndrome virus SARS-CoV-2.

### BACKGROUND OF THE INVENTION

At the end of 2019, an outbreak of atypical pneumonia of unknown etiology was recorded in Wuhan, the provincial capital of Hubei (the People's Republic of China). Studies performed by researchers have demonstrated that the outbreak was caused by a single stranded RNA virus belonging to the family Coronaviridae, lineage B betacoronaviruses (Beta-CoV B). On Feb. 11, 2020 the World Health Organization officially named the new virus SARS-CoV-2 and the disease it causes COVID-19 (“Coronavirus disease 2019”).

Within several months SARS-CoV-2 has spread around the world and has become pandemic affecting over 200 countries. By Aug. 1, 2020, the number of cases was more than 17.5 million and the number of deaths—683 thousand.

The coronavirus is spread by human-to-human transmission by respiratory droplets, dust particles in the air and direct contact. The estimated median incubation period is 5-6 days, and then the first symptoms and signs of disease develop. Common symptoms of COVID-19 include fever, dry cough, shortness of breath and fatigue. A sore throat, joint pain, runny nose, and headache have been also reported as less common symptoms.

It can be difficult to diagnose COVID-19 as its symptoms are similar to manifestations of many other viral infections. The diagnostic confirmation is based on the results of laboratory testing that require special equipment, highly skilled personnel and expensive reagents.

The clinical course of COVID-19 can vary from mild to critical cases. Severe illness is more common among people aged 60+ and patients with chronic conditions. The most serious complications of the disease include pneumonia, acute respiratory distress syndrome, acute respiratory failure, acute heart failure, acute renal failure, septic shock, cardiomyopathy, etc. However, no etiotropic drugs for use in COVID-19 treatment are currently available.

Rapid geographic spread of SARS-CoV-2 and high mortality rates have caused an urgent need to develop effective agents for the prevention of diseases caused by this virus. All research activities in this area are based on multi-year experience in the development of products directed at preventing diseases caused by other members of the Coronaviridae family.

There is a solution (patent U.S. Pat. No. 7,452,542B2) which suggests using a live, attenuated vaccine for preventing diseases caused by the coronavirus. The vaccine contains a live attenuated coronavirus wherein the virus is characterized as comprising a genome encoding an (i) ExoN polypeptide comprising a substitution at tyrosine⁶³⁹⁸ of MHV-A59, or an analogous position thereof, and (ii) Orf2a polypeptide comprising a substitution at leu¹⁰⁶ of MHV-A59, or an analogous position thereof, and a pharmaceutically acceptable solvent. Therein, the invention relates to various coronaviruses, such as avian coronavirus, animal species coronavirus and human coronavirus OC34.

There is a solution (WO2006136448A2) for obtaining a vaccine against SARS-CoV; it relates to nucleic acids encoding attenuated SARS-CoV viruses, which are capable of producing a maximum viral titer in cell culture that is reduced at least by a factor of 2 when compared to the maximum viral titer of wild-type SARS-CoV virus in the same cell culture.

There is a solution (RU 2 332 457 C2B) which for preventing coronaviral infection caused by SARS-CoV, suggests using a live bacterial vaccine wherein SARS-CoV antigens are displayed on the bacterial surface anchored with poly-gamma-glutamate synthetase (pgsBCA).

There is a solution (WO2016116398A1) which relates to the Middle East Respiratory Syndrome Coronavirus (MERS-CoV) N nucleocapsid protein and/or an immunogenic fragment thereof, or a nucleic acid molecule encoding the MERS-CoV N nucleocapsid protein and/or the immunogenic fragment thereof, for use as a vaccine. The invention further discloses the use of genetic vectors selected from the group consisting of vaccinia virus, avipoxvirus, adenovirys, alphavirys, rhabdovirus and herpesvirus for obtaining a vaccine against MERS-CoV.

There is a solution (WO2006071250A2) which suggests using vector systems comprising poxviruses and baculoviruses containing SARS-CoV S protein genes and its antigenic fragments, as a vaccine against SARS-CoV.

There is a solution (CN1276777C) which suggests using a vaccine against severe acute respiratory syndrome based on recombinant human adenovirus serotype 5 containing the SARS-CoV virus S protein sequence.

Phylogenetic analysis demonstrated that the SARS-CoV-2 virus is related to the coronaviruses found in bats (bat-SL-CoVZC45, bat-SL-CoVZXC21) more closely than the coronaviruses circulating in the human population. For instance, the S protein of SARS-CoV-2 was found to be no more than 75% homologous to the SARS-CoV S protein (Zhou P, Yang X L, Wang X G, et al. A pneumonia outbreak associated with a new coronavirus of probable bat origin. Nature. 2020;579(7798):270-273. doi:10.1038/s41586-020-2012-7). Thus, the vaccine candidates against diseases caused by SARS-CoV are not effective against COVID-19.

So far, there is no registered product for the induction of specific immunity against the SARS-CoV-2 coronavirus. As known, multiple pharmaceutical companies are developing vaccine candidates; some of them are based on the technology utilizing recombinant adenoviral vectors.

A pharmaceutical company CanSinoBIO (Tianjin, China) and the Beijing Institute of Biotechnology (Beijing, China) co-developed a recombinant adenovirus type-5 vectored (with deleted E1 and E3 regions) vaccine candidate to protect against COVID-19 which contains an optimized SARS-CoV-2 (isolate Wuhan-Hu-1) S protein gene (GenBank YP_009724390) with the tissue plasminogen activator signal peptide gene. The vaccine was manufactured as a liquid formulation containing 5×10¹⁰ viral particles per 0.5 mL. This solution was selected by the authors of the claimed invention as a prototype.

A considerable drawback of this solution is related to the fact that the vaccine could be ineffective in some groups of people due to the presence of pre-existing immunity against human adenovirus type 5.

For instance, according to the published data a single shot of this vaccine candidate was not sufficient for inducing a high level of humoral responses in people aged 55 or older (Zhu F C, Guan X H, Li Y H, et al. Immunogenicity and safety of a recombinant adenovirus type-5-vectored COVID-19 vaccine in healthy adults aged 18 years or older: a randomised, double-blind, placebo-controlled, phase 2 trial [published online ahead of print, 2020 Jul. 20]. Lancet. 2020;50140-6736(20)31605-6. doi:10.1016/S0140-6736(20)31605-6.). However, the highest risk for severe clinical course of COVID-19 is associated with the pension age.

Thus, background of the invention elicits an urgent need for developing a pharmaceutical agent that will be safe and able to induce immune response to the SARS-CoV-2 coronavirus among the broad segments of population.

### The Implementation of the Invention

The technical aim of the claimed group of inventions is to create agents for the effective induction of immune response to the SARS-CoV-2 virus.

The technical result is the creation of a safe and effective pharmaceutical agent which ensures the development of humoral and cell-mediated immune responses to the SARS-Cov-2 virus in diverse population groups, through the use of two different adenovirus vectors. Further, the technical result is the creation of a pharmaceutical agent, ensuring enhanced immune responses to the SARS-CoV-2 virus.

This technical result is achieved by that there is created a pharmaceutical agent for the induction of specific immunity against severe acute respiratory syndrome virus SARS-CoV-2, which contains component 1, comprising an agent in the form of expression vector based on a genome of recombinant human adenovirus serotype 26, wherein the E1 and E3 regions are deleted from the genome and the ORF6-Ad26 region replaced by ORF6-Ad5, with a placed expression cassette selected from SEQ ID NO:1, SEQ ID NO:2, SEQ ID NO:3, and which also contains component 2, comprising an agent in the form of expression vector based on a genome of recombinant human adenovirus serotype 5, wherein the E1 and E3 regions are deleted from the genome, with a placed expression cassette selected from SEQ ID NO:1, SEQ ID NO:2, SEQ ID NO:3 (variant 1).

Further, there is created a pharmaceutical agent for the induction of specific immunity against severe acute respiratory syndrome virus SARS-CoV-2, which contains component 1, comprising an agent in the form of expression vector based on a genome of recombinant human adenovirus serotype 26, wherein the E1 and E3 regions are deleted from the genome and the ORF6-Ad26 region replaced by ORF6-Ad5, with a placed expression cassette selected from SEQ ID NO:1, SEQ ID NO:2, SEQ ID NO:3, and which also contains component 2, comprising an agent in the form of expression vector based on a genome of recombinant simian adenovirus serotype 25, wherein the E1 and E3 regions are deleted from the genome, with a placed expression cassette selected from SEQ ID NO:4, SEQ ID NO:2, SEQ ID NO:3 (variant 2).

Furthermore, there is created a pharmaceutical agent for the induction of specific immunity against severe acute respiratory syndrome virus SARS-CoV-2, which contains component 1, comprising an agent in the form of expression vector based on a genome of recombinant simian adenovirus serotype 25, wherein the E1 and E3 regions are deleted from the genome, with a placed expression cassette selected from SEQ ID NO:4, SEQ ID NO:2, SEQ ID NO:3, and which also contains component 2, comprising an agent in the form of expression vector based on a genome of recombinant human adenovirus serotype 5, wherein the E1 and E3 regions are deleted from the genome, with a placed expression cassette selected from SEQ ID NO:1, SEQ ID NO:2, SEQ ID NO:3 (variant 3).

Therein, each of the pharmaceutical agents is presented as a liquid or lyophilized (freeze-dried) formulation.

At that, a buffer solution of the pharmaceutical agent for liquid formulation contains the following, mass %:

| | |
|---|---|
| tris | from 0.1831 to 0.3432 |
| sodium chloride | from 0.3313 to 0.6212 |
| sucrose | from 3.7821 to 7.0915 |
| magnesium chloride | from 0.0154 to 0.0289 |
| hexahydrate | |
| EDTA | from 0.0029 to 0.0054 |
| polysorbate-80 | from 0.0378 to 0.0709 |
| ethanol 95% | from 0.0004 to 0.0007 |
| water | the remaining part. |

A buffer solution of the pharmaceutical agent for lyophilized (freeze-dried) formulation contains the following, mass % %:

| | |
|---|---|
| tris | from 0.0180 to 0.0338 |
| sodium chloride | from 0.1044 to 0.1957 |
| sucrose | from 5.4688 to 10.2539 |
| magnesium chloride | from 0.0015 to 0.0028 |
| hexahydrate | |
| EDTA | from 0.0003 to 0.0005 |
| polysorbate-80 | from 0.0037 to 0.0070 |
| water | the remaining part. |

Component 1 and component 2 are placed in different packages.

Each of the pharmaceutical agents is used for inducing specific immunity against the severe acute respiratory syndrome SARS-CoV-2 virus, wherein component 1 and component 2 are used in an effective amount, sequentially, with a time interval of more than one week.

### BRIEF DESCRIPTION OF THE FIGURES

FIG. 1

presents a scheme of the expression cassette, where:

1—promoter

2—target gene,

3—polyadenylation signal.

FIG. 2

illustrates the results of effectiveness assessment of the immunization with the developed pharmaceutical agent, as estimated by the percentage of proliferating CD4+ lymphocytes re-stimulated by S glycoprotein of the SARS-CoV-2 virus at Day 8 after the immunization of experimental animals.

Y-axis—the number of proliferating cells, %

X-axis—created groups of animals:

1. Ad26-CMV-S-CoV2 (component 1), Ad5-CMV-S-CoV2 (component 2);

2. Ad26-CMV-S-CoV2 (component 1), Ad5-CAG-S-CoV2 (component 2);

3. Ad26-CMV-S-CoV2 (component 1), Ad5-EF1-S-CoV2 (component 2);

4. Ad26- CAG -S-CoV2 (component 1), Ad5-CMV-S-CoV2 (component 2);

5. Ad26- CAG -S-CoV2 (component 1), Ad5-CAG-S-CoV2 (component 2);

6. Ad26- CAG -S-CoV2 (component 1), Ad5-EF1-S-CoV2 (component 2);

7. Ad26- EF1-S-CoV2 (component 1), Ad5-CMV-S-CoV2 (component 2);

8. Ad26- EF1-S-CoV2 (component 1), Ad5-CAG-S-CoV2 (component 2);

9. Ad26- EF1-S-CoV2 (component 1), Ad5-EF1-S-CoV2 (component 2);

10. Ad26-null (component 1), Ad5-null (component 2);

11. Ad26-CMV-S-CoV2 (component 1), simAd25-CMV-S-CoV2 (component 2);

12. Ad26-CMV-S-CoV2 (component 1), simAd25-CAG-S-CoV2 (component 2);

13. Ad26-CMV-S-CoV2 (component 1), simAd25-EF1-S-CoV2 (component 2);

14. Ad26-CAG-S-CoV2 (component 1), simAd25-CMV-S-CoV2 (component 2);

15. Ad26-CAG-S-CoV2 (component 1), simAd25-CAG-S-CoV2 (component 2);

16. Ad26-CAG-S-CoV2 (component 1), simAd25-EF1-S-CoV2 (component 2);

17. Ad26-EF1-S-CoV2 (component 1), simAd25-CMV-S-CoV2 (component 2);

18. Ad26-EF1-S-CoV2 (component 1), simAd25-CAG-S-CoV2 (component 2);

19. Ad26-EF1-S-CoV2 (component 1), simAd25-EF1-S-CoV2 (component 2);

20. Ad26-null (component 1), simAd25-null (component 2);

21. simAd25-CMV-S-CoV2 (component 1), Ad5-CMV-S-CoV2 (component 2);

22. simAd25-CMV-S-CoV2 (component 1), Ad5-CAG-S-CoV2 (component 2);

23. simAd25-CMV-S-CoV2 (component 1), Ad5-EF1-S-CoV2 (component 2);

24. simAd25-CAG-S-CoV2 (component 1), Ad5-CMV-S-CoV2 (component 2);

25. simAd25-CAG-S-CoV2 (component 1), Ad5-CAG-S-CoV2 (component 2);

26. simAd25-CAG-S-CoV2 (component 1), Ad5-EF1-S-CoV2 (component 2);

27. simAd25-EF1-S-CoV2 (component 1), Ad5-CMV-S-CoV2 (component 2);

28. simAd25-EF1-S-CoV2 (component 1), Ad5-CAG-S-CoV2 (component 2);

29. simAd25-EF1-S-CoV2 (component 1), Ad5-EF1-S-CoV2 (component 2);

30. simAd25-null (component 1), Ad5-null (component 2);

31. phosphate-buffered saline

●—Data per animal

▬—Geometric mean calculated for each of the groups

FIG. 3

illustrates the results of effectiveness assessment of the immunization with the developed pharmaceutical agent, as estimated by the percentage of proliferating CD8+ lymphocytes re-stimulated by S glycoprotein of the SARS-CoV-2 virus at day 8 after the immunization of mice.

Y-axis—the number of proliferating cells, %

X-axis—created groups of animals:

1. Ad26-CMV-S-CoV2 (component 1), Ad5-CMV-S-CoV2 (component 2);

2. Ad26-CMV-S-CoV2 (component 1), Ad5-CAG-S-CoV2 (component 2);

3. Ad26-CMV-S-CoV2 (component 1), Ad5-EF1-S-CoV2 (component 2);

4. Ad26-CAG-S-CoV2 (component 1), Ad5-CMV-S-CoV2 (component 2);

5. Ad26-CAG-S-CoV2 (component 1), Ad5-CAG-S-CoV2 (component 2);

6. Ad26-CAG-S-CoV2 (component 1), Ad5-EF1-S-CoV2 (component 2);

7. Ad26-EF1-S-CoV2 (component 1), Ad5-CMV-S-CoV2 (component 2);

8. Ad26-EF1-S-CoV2 (component 1), Ad5-CAG-S-CoV2 (component 2);

9. Ad26-EF1-S-CoV2 (component 1), Ad5-EF1-S-CoV2 (component 2);

10. Ad26-null (component 1), Ad5-null (component 2);

11. Ad26-CMV-S-CoV2 (component 1), simAd25-CMV-S-CoV2 (component 2);

12. Ad26-CMV-S-CoV2 (component 1), simAd25-CAG-S-CoV2 (component 2);

13. Ad26-CMV-S-CoV2 (component 1), simAd25-EF1-S-CoV2 (component 2);

14. Ad26-CAG S-CoV2 (component 1), simAd25-CMV-S-CoV2 (component 2);

15. Ad26-CAG-S-CoV2 (component 1), simAd25-CAG-S-CoV2 (component 2);

16. Ad26-CAG-S-CoV2 (component 1), simAd25-EF1-S-CoV2 (component 2);

17. Ad26-EF1-S-CoV2 (component 1), simAd25-CMV-S-CoV2 (component 2);

18. Ad26-EF1-S-CoV2 (component 1), simAd25-CAG-S-CoV2 (component 2);

19. Ad26-EF1-S-CoV2 (component 1), simAd25-EF1-S-CoV2 (component 2);

20. Ad26-null (component 1), simAd25-null (component 2);

21. simAd25-CMV-S-CoV2 (component 1), Ad5-CMV-S-CoV2 (component 2);

22. simAd25-CMV-S-CoV2 (component 1), Ad5-CAG-S-CoV2 (component 2);

23. simAd25-CMV-S-CoV2 (component 1), Ad5-EF1-S-CoV2 (component 2);

24. simAd25-CAG-S-CoV2 (component 1), Ad5-CMV-S-CoV2 (component 2);

25. simAd25-CAG-S-CoV2 (component 1), Ad5-CAG-S-CoV2 (component 2);

26. simAd25-CAG-S-CoV2 (component 1), Ad5-EF1-S-CoV2 (component 2);

27. simAd25-EF1-S-CoV2 (component 1), Ad5-CMV-S-CoV2 (component 2);

28. simAd25-EF1-S-CoV2 (component 1), Ad5-CAG-S-CoV2 (component 2);

29. simAd25-EF1-S-CoV2 (component 1), Ad5-EF1-S-CoV2 (component 2);

30. simAd25-null (component 1), Ad5-null (component 2);

31. phosphate-buffered saline.

●—Shown data per animal

▬—Geometric mean calculated for each of the groups

FIG. 4

illustrates the survival curve of golden Syrian hamsters immunized with the developed pharmaceutical agent and control groups, using a lethal SARS-CoV-2 virus infection model.

Y-axis—animal survival rate, %

X-axis—days after challenge with the SARS-CoV-2 virus.

●—Presents the survival rate of golden Syrian hamsters immunized with the developed pharmaceutical agent, the created groups:

1) Ad26-CMV-S-CoV2 (component 1), Ad5-CMV-S-CoV2 (component 2);

2) Ad26-CMV-S-CoV2 (component 1), Ad5-CAG-S-CoV2 (component 2);

3) Ad26-CMV-S-CoV2 (component 1), Ad5-EF1-S-CoV2 (component 2);

4) Ad26-CAG-S-CoV2 component 1), Ad5-CMV-S-CoV2 (component 2);

5) Ad26-CAG-S-CoV2 (component 1), Ad5-CAG-S-CoV2 (component 2);

6) Ad26-CAG -S-CoV2 (component 1), Ad5-EF1-S-CoV2 (component 2);

7) Ad26-EF1-S-CoV2 (component 1), Ad5-CMV-S-CoV2 (component 2);

8) Ad26-EF1-S-CoV2 (component 1), Ad5-CAG-S-CoV2 (component 2);

9) Ad26-EF1-S-CoV2 (component 1), Ad5-EF1-S-CoV2 (component 2);

11) Ad26-CMV-S-CoV2 (component 1), simAd25-CMV-S-CoV2 (component 2);

12) Ad26-CMV-S-CoV2 (component 1), simAd25-CAG-S-CoV2 (component 2);

13) Ad26-CMV-S-CoV2 (component 1), simAd25-EF1-S-CoV2 (component 2);

14) Ad26-CAG-S-CoV2 (component 1), simAd25-CMV-S-CoV2 (component 2);

15) Ad26-CAG-S-CoV2 (component 1), simAd25-CAG-S-CoV2 (component 2);

16) Ad26-CAG-S-CoV2 (component 1), simAd25-EF1-S-CoV2 (component 2);

17) Ad26-EF1-S-CoV2 (component 1), simAd25-CMV-S-CoV2 (component 2);

18) Ad26-EF1-S-CoV2 (component 1), simAd25-CAG-S-CoV2 (component 2);

19) Ad26-EF1-S-CoV2 (component 1), simAd25-EF1-S-CoV2 (component 2);

21) simAd25-CMV-S-CoV2 (component 1), Ad5-CMV-S-CoV2 (component 2);

22) simAd25-CMV-S-CoV2 (component 1), Ad5-CAG-S-CoV2 (component 2);

23) simAd25-CMV-S-CoV2 (component 1), Ad5-EF1-S-CoV2 (component 2);

24) simAd25-CAG-S-CoV2 (component 1), Ad5-CMV-S-CoV2 (component 2);

25) simAd25-CAG-S-CoV2 (component 1), Ad5-CAG-S-CoV2 (component 2);

26) simAd25-CAG-S-CoV2 (component 1), Ad5-EF1-S-CoV2 (component 2);

27) simAd25-EF1-S-CoV2 (component 1), Ad5-CMV-S-CoV2 (component 2);

28) simAd25-EF1-S-CoV2 (component 1), Ad5-CAG-S-CoV2 (component 2);

29) simAd25-EF1-S-CoV2 (component 1), Ad5-EF1-S-CoV2 (component 2), which in all groups amounted to 100%.

♦—Negative control, group:

10) Ad26-null (component 1), Ad5-null (component 2).

▪—Negative control, group:

20) Ad26-null (component 1), simAd25-null (component 2).

[symbol] —Negative control, group:

30) simAd25-null (component 1), Ad5-null (component 2).

▴—Negative control, group:

32. 32)—phosphate-buffered saline.

FIG. 5

Illustratres the assessment results of humoral immune response against the SARS-CoV2 virus antigen in primates immunized with the developed pharmaceutical agent according to variant 1.

Y-axis—IgG reciprocal titer against SARS-CoV-2 RBD.

X-axis—days.

[symbol] —immunized with the developed pharmaceutical agent according to variant 1 (Ad26-CMV-S-SARS-CoV-2; Ad5-CMV-S-SARS-CoV-2).

○—Placebo.

FIG. 6

illustrates the results of effectiveness assessment of the immunization with the developed pharmaceutical agent, as estimated by the percentage of proliferating CD4+ lymphocytes re-stimulated by the RBD fragment of SARS-CoV-2 S antigen at day 8 after the immunization of primates.

Y-axis—the number of proliferating cells, %

X-axis—days.

[symbol] [symbol] [symbol] [symbol] [symbol] , [symbol] [symbol] [symbol] [symbol] [symbol] [symbol] [symbol] [symbol] 1 (Ad26-CMV-S-SARS-CoV-2; Ad5-CMV-S-SARS-CoV-2).

The black color is used to depict the group of animals immunized with the developed pharmaceutical agent according to variant 1 (Ad26-CMV-S-SARS-CoV-2; Ad5-CMV-S-SARS-CoV-2).

The control group (not vaccinated animals) is shown in grey.

Arithmetical mean value is shown as a dotted line for each of the data groups. A statistically significant difference between the values obtained for the immunized and control (not vaccinated) animals is shown by a bracket and * symbol (Mann-Whitney test, p<0.05).

FIG. 7

illustrates the results of effectiveness assessment of the immunization with the developed pharmaceutical agent, as estimated by the percentage of proliferating CD8+ lymphocytes re-stimulated by the RBD fragment of SARS-CoV-2 S antigen at day 8 after the immunization of primates.

Y-axis—the number of proliferating cells, %

X-axis—days.

The black color is used to depict the group of animals immunized with the developed pharmaceutical agent according to variant 1 (Ad26-CMV-S-SARS-CoV-2; Ad5-CMV-S-SARS-CoV-2).

The control group (not vaccinated animals) is shown in grey.

Arithmetical mean value is shown as a dotted line for each of the data groups. A statistically significant difference between the values obtained for the immunized and control (not vaccinated) animals is shown by a bracket and symbol *, p<0.05 (Mann-Whitney test).

FIG. 8

illustrates the results of effectiveness assessment of the immunization of volunteers with a liquid formulation of the developed pharmaceutical agent according to variant 1, as estimated by the percentage of proliferating CD8+ lymphocytes re-stimulated by SARS-CoV-2 S antigen.

Y-axis—the number of proliferating cells, %

X-axis—days.

●—symbols used for each of the volunteers at day 0.

▪—symbols used for each of the volunteers at day 14.

▴—symbols used for each of the volunteers at day 28.

Median value is shown as a black line for each of the data groups. A statistically significant difference between the values obtained at days 0, 14 and 28 is shown by a bracket and symbols *, p<0.05; **, p<0.01; ****, p<0.001 (Mann-Whitney test).

FIG. 9

illustrates the results of effectiveness assessment of the immunization of volunteers with a liquid formulation of the developed pharmaceutical agent according to variant 1, as estimated by the percentage of proliferating CD4+ lymphocytes re-stimulated by SARS-CoV-2 S antigen.

Y-axis—the number of proliferating cells, %

X-axis—days.

●—symbols used for each of the volunteers at day 0.

▪—symbols used for each of the volunteers at day 14.

▴—symbols used for each of the volunteers at day 28.

Median value is shown as a black line for each of the data groups. Statistically significant difference between the values obtained at days 0, 14 and 28 is shown by a bracket and symbols *, p<0.05; **, p<0.01; ****, p<0.001 (Mann-Whitney test).

FIG. 10

illustrates the results of effectiveness assessment of the immunization of volunteers with a lyophilized (freeze-dried) formulation of the developed pharmaceutical agent according to variant 1, as estimated by the percentage of proliferating CD8+ lymphocytes re-stimulated by SARS-CoV-2 S antigen.

Y-axis—the number of proliferating cells, %

X-axis—days.

●—symbols used for each of the volunteers at day 0.

▪—symbols used for each of the volunteers at day 14.

▴—symbols used for each of the volunteers at day 28.

Median value is shown as a black line for each of the data groups. Statistically significant difference between the values obtained at days 0, 14 and 28 is shown by a bracket and symbols *, p<0.05; **, p<0.01; ****, p<0.001 (Mann-Whitney test).

FIG. 11

illustrates the results of effectiveness assessment of the immunization of volunteers with a lyophilized (freeze-dried) formulation of the developed pharmaceutical agent according to variant 1, as estimated by the percentage of proliferating CD4+ lymphocytes re-stimulated by SARS-CoV-2 S antigen.

Y-axis—the number of proliferating cells, %

X-axis—days.

●—symbols used for each of the volunteers at day 0.

▪—symbols used for each of the volunteers at day 14.

▴—symbols used for each of the volunteers at day 28.

Median value is shown as a black line for each of the data groups. A statistically significant difference between the values obtained at days 0, 14 and 28 is shown by a bracket and symbols *, p<0.05; **, p<0.01; ****, p<0.001 (Mann-Whitney test).

FIG. 12

illustrates an increase in IFN [symbol] concentration (-fold) in the culture medium of peripheral blood mononuclear cells from volunteers immunized with a liquid formulation of the developed pharmaceutical agent according to variant 1, after their re-stimulation by SARS-CoV-2 S antigen prior to immunization (day 0) and at days 14 and 28 of the study.

Y-axis—increase in IFN-gamma concentration (-fold).

X-axis—days.

●—symbols used for showing values obtained for each of the volunteers at day 0.

▪—symbols used for showing values obtained for each of the volunteers at day 14.

▴—symbols used for showing values obtained for each of the volunteers at day 28.

Median value is shown as a black line for each of the data groups. A statistically significant difference between the values obtained at days 0, 14 and 28 is shown by a bracket and symbols *, p<0.05; ****, p<0.001 (Mann-Whitney test).

FIG. 13

illustrates an increase in IFN [symbol] concentration (-fold) in the culture medium of peripheral blood mononuclear cells from volunteers immunized with a lyophilized (freeze-dried) formulation of the developed pharmaceutical agent according to variant 1, after their re-stimulation by SARS-CoV-2 S antigen prior to immunization (day 0) and at days 14 and 28 of the study

Y-axis—increase in IFN-gamma concentration (-fold).

X-axis—days.

●—symbols used for showing values obtained for each of the volunteers at day 0.

▪—symbols used for showing values obtained for each of the volunteers at day 14.

▴—symbols used for showing values obtained for each of the volunteers at day 28.

Dots depict values for each of the volunteers involved in the study. Median value is shown as a black line for each of the data groups. A statistically significant difference between the values obtained at days 0, 14 and 28 is shown by a bracket and symbols *, p<0.05; ****, p<0.001 (Mann-Whitney test).

FIG. 14

illustrates the assessment results of humoral immune response against the SARS-CoV2 virus antigen in volunteers immunized with a liquid formulation of the developed pharmaceutical agent according to variant 1.

Y-axis—IgG titer against SARS-CoV-2 S glycoprotein RBD.

X-axis—days.

[symbol] —data for each of the volunteers.

FIG. 15

illustrates the assessment results of humoral immune response against the SARS-CoV2 virus antigen in volunteers immunized with a lyophilized (freeze-dried) formulation of the developed pharmaceutical agent according to variant 1.

Y-axis—IgG titer against SARS-CoV-2 S glycoprotein RBD.

X-axis—days.

[symbol] —data for each of the volunteers.

### THE IMPLEMENTATION OF THE INVENTION

To create a safe and effective pharmaceutical agent for inducing specific immune response against the SARS-CoV-2 virus, the vector system employing adenoviruses was selected. Adenoviral vectors are characterized by numerous advantages: the inability to replicate in human cells; possibility to enter both dividing and non-dividing human cells; capability to induce cell-mediated and humoral immune response; and, the potential to ensure a high level of expression of the target antigen.

At the same time, clinical applications of these vectors could be limited, as some people with adenoviral infections in the past medical history may have pre-existing immune response to adenoviruses. Research findings have demonstrated that antibody titers against an adenoviral vector increase with age and vary in different population segments. In this context, high seroprevalence levels to human adenovirus serotype 5 are reported in 40-45% of the population in the United States and up to 90% of the population in Sub-Saharan Africa. (Nwanegbo E, Vardas E, Gao W, et al. Prevalence of neutralizing antibodies to adenoviral serotypes 5 and 35 in the adult populations of The Gambia, South Africa, and the United States. Clin. Diagn. Lab. Immunol. 2004;11(2):351-357; Dudareva M, Andrews L, Gilbert S C, et al. Prevalence of serum neutralizing antibodies against chimpanzee adenovirus 63 and human adenovirus 5 in Kenyan children, in the context of vaccine vector efficacy. Vaccine. 2009;27(27):3501-3504; Zhang S, Huang W, Zhou X, Zhao Q, Wang Q, Jia B. Seroprevalence of neutralizing antibodies to human adenoviruses type-5 and type-26 and chimpanzee adenovirus type-68 in healthy Chinese adults. J. Med. Virol. 2013;85(6):1077-1084).

Neutralizing antibodies directed against vectors are responsible for a considerable reduction of specific immune response to a transgene and may reduce the effectiveness of immunization.

Based on the performed studies, the inventors identified adenoviral vector serotypes with such genetic differences that would exclude any influence on the generation of antigen-specific immune responses against the vaccine antigen during sequential immunization.

Three viruses were selected for further research—human adenovirus serotype 26, human adenovirus serotype 5 and simian adenovirus serotype 25. At the next stage, viral clones distinguished by higher growth kinetics were selected. These clones were used to create genetically engineered recombinant adenoviral vectors.

Thus, the utilized combinations of several types of the genetic vectors supported the development of a spectrum of pharmaceutical agents in order to overcome difficulties associated with the pre-existing human immune response to some adenoviruses, in particular, human adenovirus serotype 5.

With that, it is possible to use the invention wherein a variant of pharmaceutical agent is selected after the assessment of patient's immunity against adenoviral vector serotypes included in the agent formula (human adenovirus serotype 26, human adenovirus serotype 5, simian adenovirus serotype 25).

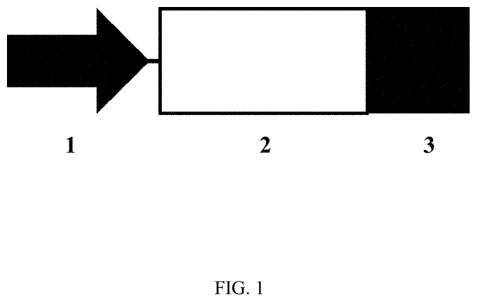

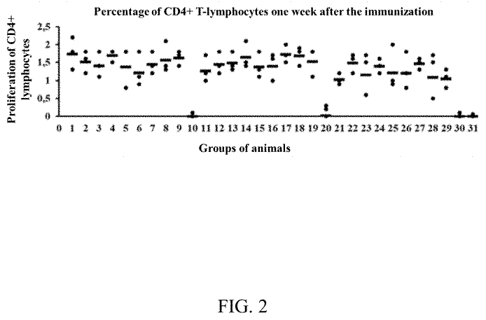

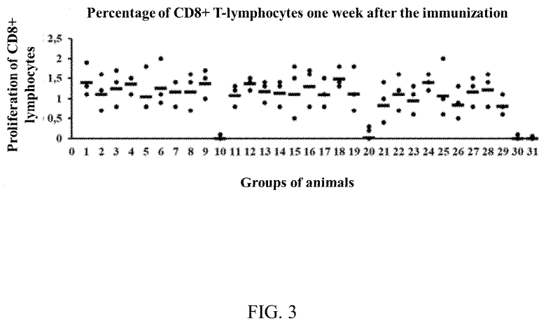

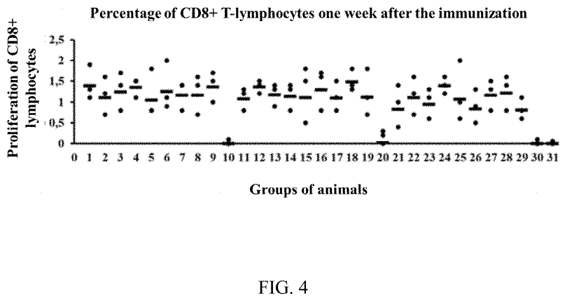

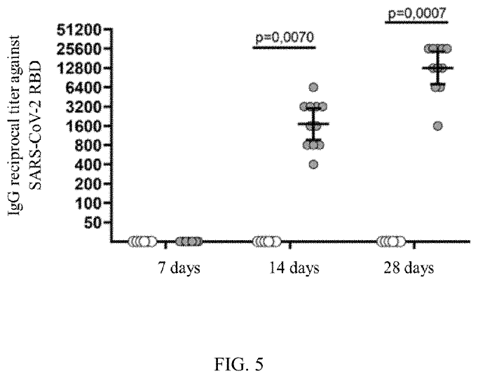

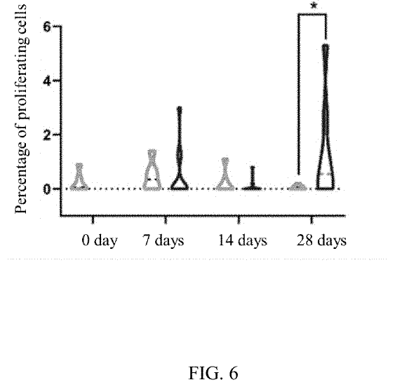

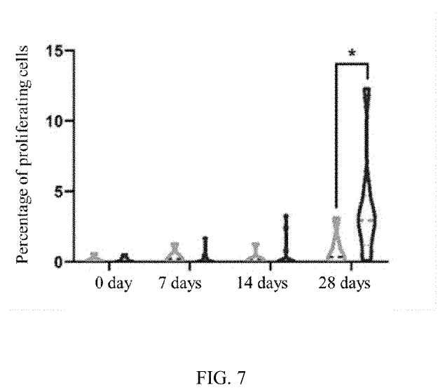

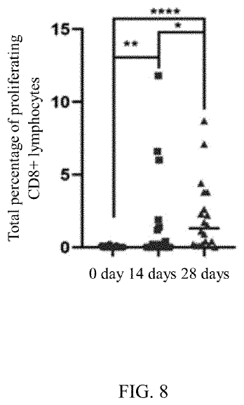

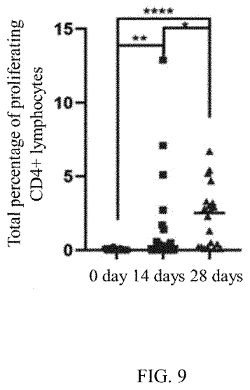

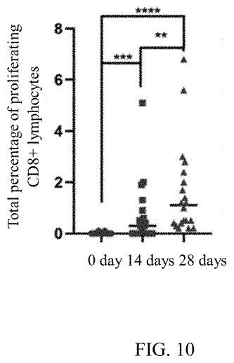

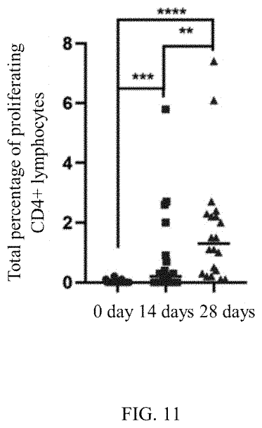

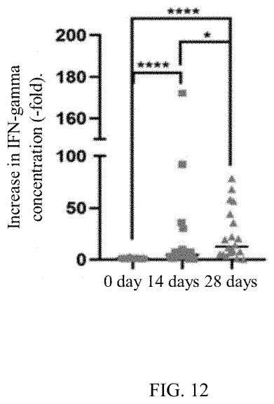

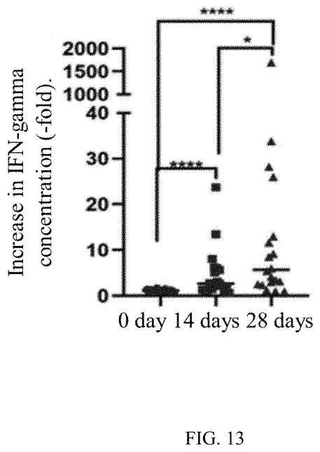

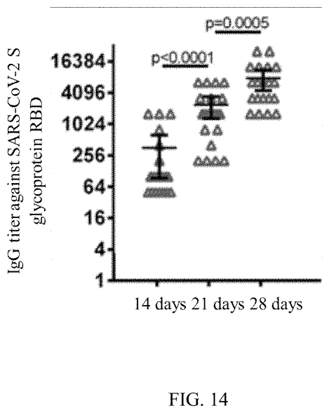

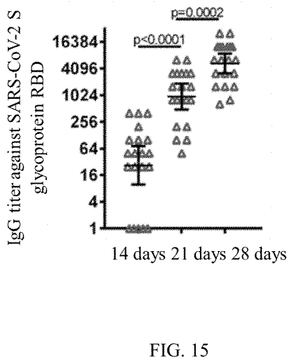

Utilizing genetic engineering techniques, expression cassettes were placed in the recombinant adenoviral vectors. The cassettes included the vaccine antigen gene and expression regulatory elements (promoter and polyadenylation signal). Schematic diagram of the expression cassette is shown on FIG. 1.

To maximize the effectiveness of induction of immune reactions, the authors claimed multiple variants of expression cassettes.

Spike (S) protein of the SARS-CoV-2 virus optimized for the expression in mammalian cells was used as an antigen in all cassettes. The S protein is one of the coronavirus structural proteins. It is exposed on the viral particle surface and is responsible for binding the virus to ACE2 (angiotensin-converting enzyme 2) receptor. The results of completed studies demonstrated the production of virus-neutralizing antibodies to the S protein, and therefore it is considered as a promising antigen for the development of pharmaceutical agents.

The expression cassette SEQ ID NO:1 contains the CMV promoter, SARS-CoV-2 virus S protein gene, and polyadenylation signal. The CMV promoter is a promoter of immediate early genes of cytomegalovirus that ensures constitutive expression in multiple cell types. However, a target-gene expression strength controlled by the CMV promoter varies for different cell types. Further, the level of transgene expression under CMV promoter control was shown to decline as the duration of cell cultivation increases. It occurs due to the suppression of gene expression relating to DNA methylation [Wang W., Jia Y L., Li Y C., Jing C Q., Guo X., Shang X F., Zhao C P., Wang T Y. Impact of different promoters, promoter mutation, and an enhancer on recombinant protein expression in CHO cells. // Scientific Reports—2017.—Vol. 8.—P. 10416].

The expression cassette SEQ ID NO:2 contains the CAG promoter, SARS-CoV-2 virus S protein gene, and polyadenylation signal. The CAG promoter is a synthetic promoter containing early enhancer of the CMV promoter, chicken β-actin promoter and chimeric intron (chicken β-actin and rabbit β-globin). Experiments demonstrated that the CAG promoter has a higher transcriptional activity compared to the CMV promoter [Yang C. Q., Li X. Y., Li Q., Fu S. L., Li H., Guo Z. K., Lin J. T., Zhao S. T. Evaluation of three different promoters driving gene expression in developing chicken embryo by using in vivo electroporation. // Genet. Mol. Res.—2014.—Vol. 13.—P. 1270-1277].

The expression cassette SEQ ID NO:3 contains the EF1 promoter, SARS-CoV-2 virus S protein gene, and polyadenylation signal. The EF1 promoter is a promoter of human eukaryotic translation elongation factor 1α (EF-1α). The promoter is constitutively active in a variety of cell types [PMID: 28557288. The EF-1α promoter maintains high-level transgene expression from episomal vectors in transfected CHO-K1 cells]. The EF-1α gene encodes the elongation factor 1α which is one of the most frequent proteins in eukaryotic cells and shows expression almost in all mammalian cell types. The EF-1α promoter frequently demonstrates its activity in the cells where viral promoters are unable to facilitate the expression of controlled genes and in the cells where viral promoters are gradually extinguished.

The expression cassette SEQ ID NO:4 contains the CMV promoter, SARS-CoV-2 virus S protein gene, and polyadenylation signal.

Thus, as a result of the accomplished task, the following 3 variants of pharmaceutical agent were developed.
1. Pharmaceutical agent for the induction of specific immunity against severe acute respiratory syndrome virus SARS-CoV-2, which contains component 1, comprising an agent in the form of expression vector based on a genome of recombinant human adenovirus serotype 26, wherein the E1 and E3 regions are deleted from the genome and the ORF6-Ad26 region replaced by ORF6-Ad5 with a placed expression cassette, selected from SEQ ID NO:1, SEQ ID NO:2, SEQ ID NO:3, and which also contains component 2, comprising an agent in the form of expression vector based on a genome of recombinant human adenovirus serotype 5, wherein the E1 and E3 regions are deleted from the genome, with a placed expression cassette selected from SEQ ID NO:1, SEQ ID NO:2, SEQ ID NO:3.
2. Pharmaceutical agent for the induction of specific immunity against severe acute respiratory syndrome virus SARS-CoV-2, which contains component 1, comprising an agent in the form of expression vector based on a genome of recombinant human adenovirus serotype 26, wherein the E1 and E3 regions are deleted from the genome and the ORF6-Ad26 region replaced by ORF6-Ad5 with a placed expression cassette selected from SEQ ID NO:1, SEQ ID NO:2, SEQ ID NO:3, and which also contains component 2, comprising an agent in the form of expression vector based on a genome of recombinant simian adenovirus serotype 25, wherein the E1 and E3 regions are deleted, from the genome with a placed expression cassette selected from SEQ ID NO:4, SEQ ID NO:2, SEQ ID NO:3
3. Pharmaceutical agent for the induction of specific immunity against severe acute respiratory syndrome virus SARS-CoV-2, which contains component 1, comprising an agent in the form of expression vector based on a genome of recombinant simian adenovirus serotype 25, wherein the E1 and E3 regions are deleted from the genome with a placed expression cassette selected from SEQ ID NO:4, SEQ ID NO:2, SEQ ID NO:3, and which also contains component 2, comprising an agent in the form of expression vector based on a genome of recombinant human adenovirus serotype 5, wherein the E1 and E3 regions are deleted from the genome, with a placed expression cassette selected from SEQ ID NO:1, SEQ ID NO:2, SEQ ID NO:3.

With that, components of the pharmaceutical agent may be placed in different packages.

Further, the authors of the invention have developed liquid and lyophilized (freeze-dried) formulations of the pharmaceutical agent.

Furthermore, the inventors selected such variants of the buffer solution that allow storing the developed pharmaceutical agent both frozen at a temperature below −18° C. and lyophilized (freeze-dried) at a temperature range from +2° C. to +8° C.

Also, a method of utilization of the pharmaceutical agent was developed for inducing specific immunity against the severe acute respiratory syndrome SARS-CoV-2 virus, wherein component 1 and component 2 are used in effective amount, sequentially, with a time interval of more than one week.
The implementation of the invention is proven by the following examples:

### EXAMPLE 1

### Production of the Expression Vector Containing the Genome of Recombinant Human Adenovirus Serotype 26

At the first stage, a design of plasmid construction pAd26-Ends was proposed. It carries the two regions homologous to the genome of recombinant human adenovirus serotype 26 (two homology arms) and the ampicillin-resistance gene. One of the homology arms is a beginning portion of the genome of recombinant human adenovirus serotype 26 (from the left inverted terminal repeat to the E1 region) and sequence of the viral genome including pIX protein). The other homology arm contains a nucleotide sequence located after ORF3 E4 region through the end of the genome. Synthesis of pAd26-Ends construction was performed by the Moscow company “Eurogen” ZAO.

Human adenovirus serotype 26 DNA isolated from virions was mixed with pAd26-Ends. A plasmid pAd26-dlE1, carrying the genome of human adenovirus serotype 26 with the deleted E1 region, was obtained through the process of homologous recombination between pAd26-Ends and viral DNA.

Then, in the obtained plasmid pAd26-dlE1, using standard cloning techniques, the sequence containing an open reading frame 6 (ORF6-Ad26) was replaced with a similar sequence from the genome of human adenovirus serotype 5. The aim of this manipulation was to ensure that human adenovirus serotype 26 is capable to replicate effectively in HEK293 cell culture. As a result, the plasmid pAd26-dlE1-ORF6-Ad5 was derived.

Further, using standard genetic engineering techniques, the E3 region (approx. 3321 base pairs between the genes pVIII and U-exon) of the adenoviral genome was deleted from the constructed plasmid pAd26-dlE1-ORF6-Ad5 in order to expand packaging capacity of the vector. Ultimately, a recombinant vector pAd26-only-null based on the genome of human adenovirus serotype 26 with the open reading frame ORF6 of human adenovirus serotype 5 and with deleted E1 and E3 regions was obtained. The sequence SEQ ID NO:5 was utilized as a parental sequence of human adenovirus serotype 26.

Also, the authors developed multiple designs of expression cassette:
expression cassette SEQ ID NO:1 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Based on the plasmid construction pAd26-Ends, utilizing genetic engineering techniques, constructions pArms-26-CMV-S-CoV2, pArms-26-CAG-S-CoV2, pArms-26-EF1-S-CoV2 were obtained. The latter constructions contain the expression cassettes SEQ ID NO:1, SEQ ID NO:2 or SEQ ID NO:3, respectively, as well as carrying homology arms of the genome of human adenovirus serotype 26.

Next, the constructions pArms-26-CMV-S-CoV2, pArms-26-CAG-S-CoV2, pArms-26-EF1-S-CoV2 were linearized by a unique hydrolysis site between the homology arms; each of the plasmids was mixed with the recombinant vector pAd26-only-null.

The homologous recombination allowed obtaining the plasmids pAd26-only-CMV-S-CoV2, pAd26-only-CAG-S-CoV2, pAd26-only-EF1-S-CoV2 which carry the genome of recombinant human adenovirus serotype 26 with the open reading frame ORF6 of human adenovirus serotype 5 and the deletion of E1 and E3 regions, with the expression cassette SEQ ID NO:1, SEQ ID NO:2 or SEQ ID NO:3, respectively.

During the fourth stage, the plasmids pAd26-only-CMV-S-CoV2, pAd26-only-CAG-S-CoV2, pAd26-only-EF1-S-CoV2 were hydrolyzed with the specific restriction endonucleases to remove the vector part. The derived DNA products were used for the transfection of HEK293 cell culture.

Thus, an expression vector was obtained which contains the genome of recombinant human adenovirus serotype 26, wherein the E1 and E3 regions are deleted and RF6-Ad26 region is replaced by ORF6-Ad5, with an integrated expression cassette selected from SEQ ID NO:1, SEQ ID NO:2, SEQ ID NO:3.

### EXAMPLE 2

Production of an immunobiological agent in the form of expression vector based on the genome of recombinant human adenovirus serotype 26 wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3.

At this stage, the expression vectors obtained in Example 1 were purified using anion-exchange and exclusion chromatography. The finished suspension contained adenoviral particles in the buffer solution for a liquid formulation of the pharmaceutical agent or in the buffer solution for a lyophilized (freeze-dried) formulation of the pharmaceutical agent.

Thus, the following immunobiological agents were produced on the basis of the genome of recombinant human adenovirus serotype 26, wherein the E1 and E3 regions are deleted and the ORF6-Ad26 region is replaced by ORF6-Ad5:
1. Immunobiological agent based on the genome of recombinant human adenovirus serotype 26, wherein the E1 and E3 regions are deleted and the ORF6-Ad26 region is replaced by ORF6-Ad5, with the expression cassette, containing the CMV promoter, SARS-CoV-2 virus S protein gene, and polyadenylation signal, SEQ ID NO:1 (Ad26-CMV-S-CoV2) in the buffer solution for a liquid formulation of the pharmaceutical agent.
2. Immunobiological agent based on the genome of recombinant human adenovirus serotype 26, wherein the E1 and E3 regions are deleted and the ORF6-Ad26 region is replaced by ORF6-Ad5, with the expression cassette, containing the CMV promoter, SARS-CoV-2 virus S protein gene, and polyadenylation signal, SEQ ID NO:1 (Ad26-CMV-S-CoV2) in the buffer solution for a lyophilized (freeze-dried) formulation of the pharmaceutical agent.
3. Immunobiological agent based on the genome of recombinant human adenovirus serotype 26, wherein the E1 and E3 regions are deleted and the ORF6-Ad26 region is replaced by ORF6-Ad5, with the expression cassette, containing the CAG promoter, SARS-CoV-2 virus S protein gene, and polyadenylation signal, SEQ ID NO:2 (Ad26-CAG-S-CoV2) in the buffer solution for a liquid formulation of the pharmaceutical agent.
4. Immunobiological agent based on the genome of recombinant human adenovirus serotype 26, wherein the E1 and E3 regions are deleted and the ORF6-Ad26 region is replaced by ORF6-Ad5, with the expression cassette, containing the CAG promoter, SARS-CoV-2 virus S protein gene, and polyadenylation signal, SEQ ID NO:2 (Ad26-CAG-S-CoV2) in the buffer solution for a lyophilized (freeze-dried) formulation of the pharmaceutical agent.
5. Immunobiological agent based on the genome of recombinant human adenovirus serotype 26, wherein the E1 and E3 regions are deleted and the ORF6-Ad26 region is replaced by ORF6-Ad5, with the expression cassette, containing the EF1 promoter, SARS-CoV-2 virus S protein gene, and polyadenylation signal, SEQ ID NO:3 (Ad26-EF1-S-CoV2) in the buffer solution for a liquid formulation of the pharmaceutical agent.
6. Immunobiological agent based on the genome of recombinant human adenovirus serotype 26, wherein the E1 and E3 regions are deleted and the ORF6-Ad26 region is replaced by ORF6-Ad5, with the expression cassette, containing the EF1 promoter, SARS-CoV-2 virus S protein gene, and polyadenylation signal, SEQ ID NO:3 (Ad26-EF1-S-CoV2) in the buffer solution for a lyophilized (freeze-dried) formulation of the pharmaceutical agent.

Each of the presented immunobiological agents is a component 1 in variant 1 and variant 2 of the developed pharmaceutical agent.

### EXAMPLE 3

### Production of the Expression Vector Containing the Genome of Recombinant Simian Adenovirus Serotype 25

At the first stage, a design of plasmid construction pSim25-Ends was proposed. It carries two regions homologous to the genome of simian adenovirus serotype 25 (two homology arms). One of the homology arms is a beginning portion of the genome of simian adenovirus serotype 25 (from the left inverted terminal repeat to the E1 region) and sequence from the end of the E1 region to pIVa2 protein. The other homology arm contains the sequence of the end of adenovirus genome, including the right inverted terminal repeat. Synthesis of pSim25-Ends construction was performed by the Moscow company “Eurogen” ZAO.

The DNA of simian adenovirus serotype 25 isolated from virions was mixed with pSim25-Ends. A plasmid pSim25-dlE1, carrying the genome of simian adenovirus serotype 25 with deleted E1 region, was obtained through the process of homologous recombination between pSim25-Ends and viral DNA.

Further, using standard genetic engineering techniques, the E3 region (approx. 3921 base pairs from the beginning portion of gene 12,5 [symbol] to gene 14.7K) of the adenoviral genome was deleted from the constructed plasmid pSim25-dlE1 in order to expand packaging capacity of the vector. Ultimately, a plasmid construction pSim25-null, encoding the full genome of simian adenovirus serotype 25 with deleted E1 and E3 regions was obtained. The sequence SEQ ID NO:6 was utilized as a parental sequence of simian adenovirus serotype 25.

Also, the authors developed multiple designs of expression cassette:
expression cassette SEQ ID NO:4 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Then, based on the plasmid construction pSim25-Ends, utilizing genetic engineering techniques, constructions pArms-Sim25-CMV-S-CoV2, pArms-Sim25-CAG-S-CoV2, pArms-Sim25-EF1-S-CoV2 were obtained. The latter constructions contain the expression cassettes SEQ ID SEQ ID NO:4, SEQ ID NO:2 or SEQ ID NO:3, respectively, as well as carrying homology arms from the genome of simian adenovirus serotype 25. Next, the constructions pArms-Sim25-CMV-S-CoV2, pArms-Sim25-CAG-S-CoV2, pArms-Sim25-EF1-S-CoV2 were linearized by a unique hydrolysis site between the homology arms; each of the plasmids was mixed with the recombinant vector pSim25-null. The homologous recombination allowed obtaining plasmid vectors pSim25-CMV-S-CoV2, pSim25-CAG-S-CoV2, pSim25-EF1-S-CoV2, which contain the full genome of recombinant human adenovirus serotype 26 with the open reading frame ORF6 of simian adenovirus serotype 25 with deleted E1 and E3 regions, and the expression cassette SEQ ID NO:4, SEQ ID NO:2 or SEQ ID NO:3, respectively.

During the third stage, the plasmids pSim25-CMV-S-CoV2, pSim25-CAG-S-CoV2, pSim25-EF1-S-CoV2 were hydrolyzed with the specific restriction endonuclease to remove the vector part. The derived DNA products were used for the transfection of HEK293 cell culture. The produced material was used for generating preparative amounts of the recombinant adenoviruses.

As a result, recombinant human adenoviruses serotype 25 were obtained which contain SARS-CoV-2 virus S protein gene; simAd25-CMV-S-CoV2 (containing expression cassette SEQ ID NO:2), simAd25-EF1-S-CoV2 (containing expression cassette SEQ ID NO:3).

Thus, an expression vector was obtained which contains the genome of recombinant simian adenovirus 25, wherein the E1 and E3 regions are deleted, with an integrated expression cassette selected from SEQ ID NO:4, SEQ ID NO:2, SEQ ID NO:3.

### EXAMPLE 4

Production of an immunobiological agent in the form of expression vector based on the genome of recombinant simian adenovirus serotype 25, wherein the E1 and E3 regions are deleted, with an integrated expression cassette selected from SEQ ID NO:1, SEQ ID NO:2, SEQ ID NO:3.

At this stage, the expression vectors obtained in Example 3 were purified using anion-exchange and exclusion chromatography. The finished suspension contained adenoviral particles in buffer solution for a liquid formulation of the pharmaceutical agent or in buffer solution for a lyophilized (freeze-dried) formulation of the pharmaceutical agent.

Thus, the following immunobiological agents were produced on the basis of the genome of simian adenovirus serotype 25, wherein the E1 and E3 regions are deleted:
1. Immunobiological agent based on the genome of recombinant simian adenovirus serotype 25, wherein the E1 and E3 regions are deleted, with the expression cassette, containing the CMV promoter, SARS-CoV-2 virus S protein gene, and polyadenylation signal, SEQ ID NO:1 (simAd25-CMV-S-CoV2) in the buffer solution for a liquid formulation of the pharmaceutical agent.
2. Immunobiological agent based on the genome of recombinant simian adenovirus serotype 25, wherein the E1 and E3 regions are deleted, with the expression cassette, containing the CMV promoter, SARS-CoV-2 virus S protein gene, and polyadenylation signal, SEQ ID NO:1 (simAd25-CMV-S-CoV2) in the buffer solution for a lyophilized (freeze-dried) formulation of the pharmaceutical agent.
3. Immunobiological agent based on the genome of recombinant simian adenovirus serotype 25, wherein the E1 and E3 regions are deleted, with the expression cassette, containing the CAG promoter, SARS-CoV-2 virus S protein gene, and polyadenylation signal, SEQ ID NO:2 (simAd25-CAG-S-CoV2) in the buffer solution for a liquid formulation of the pharmaceutical agent.
4. Immunobiological agent based on the genome of recombinant simian adenovirus serotype 25, wherein the E1 and E3 regions are deleted, with the expression cassette, containing the CAG promoter, SARS-CoV-2 virus S protein gene, and polyadenylation signal, SEQ ID NO:2 (simAd25-CAG-S-CoV2) in the buffer solution for a lyophilized (freeze-dried) formulation of the pharmaceutical agent.
5. Immunobiological agent based on the genome of recombinant simian adenovirus serotype 25, wherein the E1 and E3 regions are deleted, with the expression cassette, containing the EF1 promoter, SARS-CoV-2 virus S protein gene, and polyadenylation signal, SEQ ID NO:3 (simAd25-EF1-S-CoV2) in the buffer solution for a liquid formulation of the pharmaceutical agent.
6. Immunobiological agent based on the genome of recombinant simian adenovirus serotype 25, wherein the E1 and E3 regions are deleted, with the expression cassette, containing the EF1 promoter, SARS-CoV-2 virus S protein gene, and polyadenylation signal, SEQ ID NO:3 (simAd25-EF1-S-CoV2) in the buffer solution for a lyophilized (freeze-dried) formulation of the pharmaceutical agent.

Each of the presented immunobiological agents comprises component 2 in variant 1 of the developed pharmaceutical agent and component 1 in variant 3 of the developed pharmaceutical agent.

### EXAMPLE 5

### Production of the Expression Vector Containing the Genome of Recombinant Human Adenovirus Serotype 5

At the first stage, a design of plasmid construction pAd5-Ends was proposed. It carries two regions homologous to the genome of recombinant human adenovirus serotype 5 (two homology arms). One of the homology arms is a beginning portion of the genome of recombinant human adenovirus serotype 5 (from the left inverted terminal repeat to the E1 region) and sequence of the viral genome including pIX protein. The other homology arm contains a nucleotide sequence located after the ORF3 E4 region through the end of the genome. Synthesis of pAd26-Ends construction was performed by the Moscow company “Eurogen” ZAO.

Human adenovirus serotype 5 DNA isolated from virions was mixed with pAd26-Ends. A plasmid pAd26-d1E1, carrying the genome of human adenovirus serotype 5 with the deleted E1 region, was obtained through the process of homologous recombination between pAd26-Ends and viral DNA.

Further, using standard genetic engineering techniques, the E3 region of the adenoviral genome (2685 base pairs from the end of gene 12.5 [symbol] to the beginning portion of the sequence of U-exon) was deleted from the constructed plasmid pAd5-dlE1 in order to expand packaging capacity of the vector. Ultimately, a recombinant plasmid vector pAd5-too-null based on the genome of human adenovirus serotype 5 with deletions of the E1 and E3 regions was obtained. The sequence SEQ ID NO:7 was utilized as a parental sequence of human adenovirus serotype 5.

Also, the authors developed multiple designs of the expression cassette:
expression cassette SEQ ID NO:1 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Then, based on the plasmid construction pAd5-Ends, utilizing genetic engineering techniques, pArms-Ad5-CMV-S-CoV2, pArms-Ad5-CAG-S-CoV2, pArms-Ad5-EF1-S-CoV2 were obtained. The latter constructions contain the expression cassettes SEQ ID NO:1, SEQ ID NO:2 or SEQ ID NO:3, respectively, as well as carrying homology arms of the genome of human adenovirus serotype 5.

Next, the constructions pArms-Ad5-CMV-S-CoV2, pArms-Ad5-CAG-S-CoV2, pArms-Ad5-EF1-S-CoV2 were linearized by a unique hydrolysis site between homology arms; each of the plasmids was mixed with the recombinant vector pAd5-too-null. The homologous recombination allowed obtaining plasmids pAd5-too-CMV-S-CoV2, pAd5-too-GAC-S-CoV2, pAd5-too-EF1-S-CoV2, carrying the genome of recombinant human adenovirus serotype 5 with the deletion of the E1 and E3 regions, and the expression cassettes SEQ ID NO:1, SEQ ID NO:2 or SEQ ID NO:3, respectively.

During the fourth stage, the plasmids pAd5-too-CMV-S-CoV2, pAd5-too-GAC-S-CoV2, pAd5-too-EF1-S-CoV2 were hydrolyzed with the specific restriction endonuclease to remove the vector part. The derived DNA product was used for the transfection of HEK293 cell culture. The produced material was used for accumulating preparative amounts of the recombinant adenovirus.

As a result, recombinant human adenoviruses serotype 5 were obtained which include SARS-CoV-2 virus S protein gene; Ad5-CMV-S-CoV2 (containing expression cassette SEQ ID NO:1), Ad5-CAG-S-CoV2 (containing expression cassette SEQ ID NO:2), Ad5-EF1-S-CoV2 (containing expression cassette SEQ ID NO:3).

Thus, an expression vector was obtained which contains the genome of recombinant human adenovirus 5, wherein the E1 and E3 regions are deleted, with an integrated expression cassette selected from SEQ ID NO:1, SEQ ID NO:2, SEQ ID NO:3.

### EXAMPLE 6

Production of an immunobiological agent in the form of expression vector based on the genome of recombinant human adenovirus serotype 5, wherein the E1 and E3 regions are deleted, with an integrated expression cassette selected from SEQ ID NO:1, SEQ ID NO:2, SEQ ID NO:3.

At this stage, the expression vectors obtained in Example 5 were purified using anion-exchange and exclusion chromatography. The finished suspension contained adenoviral particles in the buffer solution for a liquid formulation of the pharmaceutical agent or in the buffer solution for a lyophilized (freeze-dried) formulation of the pharmaceutical agent.

Thus, the following immunobiological agents were produced on the basis of the genome of recombinant human adenovirus serotype 5, wherein the E1 and E3 regions are deleted:
1. Immunobiological agent based on the genome of recombinant human adenovirus serotype 5, wherein the E1 and E3 regions are deleted, with the expression cassette, containing the CMV promoter, SARS-CoV-2 virus S protein gene, and polyadenylation signal, SEQ ID NO:1 (Ad5-CMV-S-CoV2) in the buffer solution for a liquid formulation of the pharmaceutical agent.
2. Immunobiological agent based on the genome of recombinant human adenovirus serotype 5, wherein the E1 and E3 regions are deleted, with the expression cassette, containing the CMV promoter, SARS-CoV-2 virus S protein gene, and polyadenylation signal, SEQ ID NO:1 (Ad5-CMV-S-CoV2) in the buffer solution for a lyophilized (freeze-dried) formulation of the pharmaceutical agent.
3. Immunobiological agent based on the genome of recombinant human adenovirus serotype 5, wherein the E1 and E3 regions are deleted, with the expression cassette, containing the CAG promoter, SARS-CoV-2 virus S protein gene, and polyadenylation signal, SEQ ID NO:2 (Ad5-CAG-S-CoV2) in the buffer solution for a liquid formulation of the pharmaceutical agent.
4. Immunobiological agent based on the genome of recombinant human adenovirus serotype 5, wherein the E1 and E3 regions are deleted, with the expression cassette, containing the CAG promoter, SARS-CoV-2 virus S protein gene, and polyadenylation signal, SEQ ID NO:2 (Ad5-CAG-S-CoV2) in the buffer solution for a lyophilized (freeze-dried) formulation of the pharmaceutical agent.
5. Immunobiological agent based on the genome of recombinant human adenovirus serotype 5, wherein the E1 and E3 regions are deleted, with the expression cassette, containing the EF1 promoter, SARS-CoV-2 virus S protein gene, and polyadenylation signal, SEQ ID NO:3 (Ad5-EF1-S-CoV2) in the buffer solution for a liquid formulation of the pharmaceutical agent.
6. Immunobiological agent based on the genome of recombinant human adenovirus serotype 5, wherein the E1 and E3 regions are deleted, with the expression cassette, containing the EF1 promoter, SARS-CoV-2 virus S protein gene, and polyadenylation signal, SEQ ID NO:3 (Ad5-EF1-S-CoV2) in the buffer solution for a lyophilized (freeze-dried) formulation of the pharmaceutical agent.

Each of the presented immunobiological agents comprises component 1 in variant 1 and in variant 2 of the developed pharmaceutical agent.

Each of the presented immunobiological agents comprises component 2 in variant 1 and in variant 3 of the developed pharmaceutical agent.

### EXAMPLE 7

### Production of Buffer Solution

The pharmaceutical agent developed according to the present invention, consists of two components placed in different vials. With that, every component comprises immunobiological agent based on the recombinant adenovirus with the expression cassette in buffer solution.

The inventors have selected composition of the buffer solution ensuring stability of the recombinant viral particles. The solution includes:
1. Tris(hydroxymethyl)aminomethane (Tris) required for maintaining the solution pH value.
2. Sodium chloride added for reaching the necessary ionic force and osmolarity
3. Sucrose used as a cryoprotectant.
4. Magnesium chloride hexahydrate required as a source of bivalent cations.
5. EDTA used as an inhibitor of free-radical oxidation.
6. Polysorbate-80 used as a source of surfactant.
7. Ethanol 95% used as an inhibitor of free-radical oxidation.
8. Water used as a solvent.

The authors of the invention developed two variants of the buffer solution: for liquid formulation of the pharmaceutical agent and for lyophilized (freeze-dried) formulation of the pharmaceutical agent.

For estimating concentrations of the substances included in the composition of the buffer solution for liquid formulation of the pharmaceutical agent, several options of experimental groups were produced (Table 1). One of the components of the pharmaceutical agent was added to each of the produced buffer solutions:
1. Immunobiological agent based on the genome of recombinant human adenovirus serotype 26 with the expression cassette, containing the CMV promoter, SARS-CoV-2 virus S protein gene, and polyadenylation signal, 1*10¹¹ viral particles.
2. Immunobiological agent based on the genome of recombinant human adenovirus serotype 5 with the expression cassette, containing the CMV promoter, SARS-CoV-2 virus S protein gene, and polyadenylation signal, 1*10¹¹ viral particles.
3. Immunobiological agent based on the genome of recombinant simian adenovirus serotype 25 with the expression cassette, containing the CMV promoter, SARS-CoV-2 virus S protein gene, and polyadenylation signal, 1*10¹¹ viral particles.

Thus, the stability of each of the adenoviral serotypes included in the pharmaceutical agent formula was verified. The obtained pharmaceutical agents were stored at −18° C. and −70° C. for 3 months and then thawed; and changes of the titers of recombinant adenoviruses were assessed.

TABLE 1
Composition of experimental buffer solutions for liquid formulation of the
pharmaceutical agent

| | Composition of buffer solution | | | | | | | |
|---|---|---|---|---|---|---|---|---|
| | | Sodium | | Magnesium | | | Ethanol | |
| Group | Tris | chloride | Sucrose | chloride | EDTA | Polysorbate- | 95% | |
| No. | (mg) | (mg) | (mg) | hexahydrate (mg) | (mg) | 80 (mg) | (mg) | Water |
| 1 | 0.968 | 2.19 | 25 | 0.102 | 0.019 | 0.25 | 0.0025 | to 0.5 |
| | | | | | | | | ml |
| 2 | 1.815 | 2.19 | 25 | 0.102 | 0.019 | 0.25 | 0.0025 | to 0.5 |
| | | | | | | | | ml |
| 3 | 1.21 | 1.752 | 25 | 0.102 | 0.019 | 0.25 | 0.0025 | to 0.5 |
| | | | | | | | | ml |
| 4 | 1.21 | 3.285 | 25 | 0.102 | 0.019 | 0.25 | 0.0025 | to 0.5 |
| | | | | | | | | ml |
| 5 | 1.21 | 2.19 | 20 | 0.102 | 0.019 | 0.25 | 0.0025 | to 0.5 |
| | | | | | | | | ml |
| 6 | 1.21 | 2.19 | 37.5 | 0.102 | 0.019 | 0.25 | 0.0025 | to 0.5 |
| | | | | | | | | ml |
| 7 | 1.21 | 2.19 | 25 | 0.0816 | 0.019 | 0.25 | 0.0025 | to 0.5 |
| | | | | | | | | ml |
| 8 | 1.21 | 2.19 | 25 | 0.153 | 0.019 | 0.25 | 0.0025 | to 0.5 |
| | | | | | | | | ml |
| 9 | 1.21 | 2.19 | 25 | 0.102 | 0.0152 | 0.25 | 0.0025 | to 0.5 |
| | | | | | | | | ml |
| 10 | 1.21 | 2.19 | 25 | 0.102 | 0.0285 | 0.25 | 0.0025 | to 0.5 |
| | | | | | | | | ml |
| 11 | 1.21 | 2.19 | 25 | 0.102 | 0.019 | 0.2 | 0.0025 | to 0.5 |
| | | | | | | | | ml |
| 12 | 1.21 | 2.19 | 25 | 0.102 | 0.019 | 0.375 | 0.0025 | to 0.5 |
| | | | | | | | | ml |
| 13 | 1.21 | 2.19 | 25 | 0.102 | 0.019 | 0.25 | 0.002 | to 0.5 |
| | | | | | | | | ml |
| 14 | 1.21 | 2.19 | 25 | 0.102 | 0.019 | 0.25 | 0.00375 | to 0.5 |
| | | | | | | | | ml |
| 15 | 1.21 | 2.19 | 25 | 0.102 | 0.019 | 0.25 | 0.0025 | to 0.5 |
| | | | | | | | | ml |

The results of the performed experiments demonstrated that the titer of recombinant adenoviruses did not change after their storage in the buffer solution for liquid formulation of the pharmaceutical agent at a temperature of −18° C. and −70° C. for 3 months.

Thus, the developed buffer solution for liquid formulation of the pharmaceutical agent ensures the stability of all components of the developed pharmaceutical agent in the following range of active moieties (mass %):
Tris: from 0.1831 mass % to 0.3432 mass %;
Sodium chloride: from 0.3313 mass % to 0.6212 mass %;
Sucrose: from 3.7821 mass % to 7.0915 mass %;
Magnesium chloride hexahydrate: from 0.0154 mass % to 0.0289 mass %;
EDTA: from 0.0029 mass % to 0.0054 mass %;
Polysorbate-80: from 0.0378 mass % to 0.0709 mass %;
Ethanol 95%: from 0.0004 mass % to 0.0007 mass %;
Solvent: the remaining part.

For estimating concentrations of the substances included in the composition of the buffer solution for lyophilized (freeze-dried) formulation of the pharmaceutical agent, several options of experimental groups were proposed (Table 2). One of the components of the pharmaceutical agent was added to each of the produced buffer solutions:
1. Immunobiological agent based on the genome of recombinant human adenovirus serotype 26 with the expression cassette, containing the CMV promoter, SARS-CoV-2 virus S protein gene, and polyadenylation signal, 1*10¹¹ viral particles.
2. Immunobiological agent based on the genome of recombinant human adenovirus serotype 5 with the expression cassette, containing the CMV promoter, SARS-CoV-2 virus S protein gene, and polyadenylation signal, 1*10¹¹ viral particles.
3. Immunobiological agent based on the genome of recombinant simian adenovirus serotype 25 with the expression cassette, containing the CMV promoter, SARS-CoV-2 virus S protein gene, and polyadenylation signal, 1*10¹¹ viral particles.

Thus, the stability of each of the adenoviral serotypes included in the pharmaceutical agent formula was verified. The obtained pharmaceutical agents were stored at +2° C. and +8° C. for 3 months and then thawed; and changes of the titers of recombinant adenoviruses were assessed.

TABLE 2
Composition of experimental buffer solutions

| | Composition of buffer solution | | | | | | |
|---|---|---|---|---|---|---|---|
| | | | | Magnesium | | | |
| | | Sodium | | chloride | | | |
| Group | Tris | chloride | Sucrose | hexahydrate | EDTA | Polysorbate- | |
| No. | (mg) | (mg) | (mg) | (mg) | (mg) | 80 (mg) | Water |
| 1 | 0.1936 | 1.403 | 73.5 | 0.0204 | 0.0038 | 0.05 | to 1 ml |
| 2 | 0.363 | 1.403 | 73.5 | 0.0204 | 0.0038 | 0.05 | to 1 ml |
| 3 | 0.242 | 1.1224 | 73.5 | 0.0204 | 0.0038 | 0.05 | to 1 ml |
| 4 | 0.242 | 2.1045 | 73.5 | 0.0204 | 0.0038 | 0.05 | to 1 ml |
| 5 | 0.242 | 1.403 | 58.8 | 0.0204 | 0.0038 | 0.05 | to 1 ml |
| 6 | 0.242 | 1.403 | 110.25 | 0.0204 | 0.0038 | 0.05 | to 1 ml |
| 7 | 0.242 | 1.403 | 73.5 | 0.01632 | 0.0038 | 0.05 | to 1 ml |
| 8 | 0.242 | 1.403 | 73.5 | 0.0306 | 0.0038 | 0.05 | to 1 ml |
| 9 | 0.242 | 1.403 | 73.5 | 0.0204 | 0.00304 | 0.05 | to 1 ml |
| 10 | 0.242 | 1.403 | 73.5 | 0.0204 | 0.0057 | 0.05 | to 1 ml |
| 11 | 0.242 | 1.403 | 73.5 | 0.0204 | 0.0038 | 0.04 | to 1 ml |
| 12 | 0.242 | 1.403 | 73.5 | 0.0204 | 0.0038 | 0.075 | to 1 ml |
| 13 | 0.242 | 1.403 | 73.5 | 0.0204 | 0.0038 | 0.05 | to 1 ml |

The results of the performed experiments demonstrated that the titer of recombinant adenoviruses did not change after their storage in the buffer solution for lyophilized (freeze-dried) formulation of the pharmaceutical agent at a temperature of +2° C. and +8° C. for 3 months.

Thus, the developed buffer solution for lyophilized (freeze-dried) formulation of the pharmaceutical agent ensures the stability of all components of the developed pharmaceutical agent in the following range of active moieties:
Tris: from 0.0180 mass % to 0.0338 mass %;
Sodium chloride: from 0.1044 mass % to 0.1957 mass %;
Sucrose: from 5.4688 mass % to 10.2539 mass %;
Magnesium chloride hexahydrate: from 0.0015 mass % to 0.0028 mass %;
EDTA: from 0.0003 mass % to 0.0005 mass. %;
Polysorbate-80: from 0.0037 mass % to 0.0070 mass %;
Solvent: the remaining part.

### EXAMPLE 8

### Assessment of the Effectiveness of Immunization with the Developed Pharmaceutical Agent Based on the Evaluation of Humoral Immune Response

One of the key characteristics of the effectiveness of immunization is the antibody titer. The example elicits the data relating to the changes in antibody titers against SARS-CoV-2 glycoprotein at day 21 following the administration of the pharmaceutical agent to laboratory animals.

The mammalian species—BALB/c mice, females weighing 18 g were used in the experiment. All animals were divided into 31 groups, 5 animals per group, to whom component 1 of the pharmaceutical agent was injected intramuscularly at a dose 10⁸ viral particles/100 μl and two weeks later—component 2 at a dose 10⁸ viral particles/100 μl. Thus, the following groups of animals were formed:
1) Ad26-CMV-S-CoV2 (component 1), Ad5-CMV-S-CoV2 (component 2);
2) Ad26-CMV-S-CoV2 (component 1), Ad5-CAG-S-CoV2 (component 2);
3) Ad26-CMV-S-CoV2 (component 1), Ad5-EF1-S-CoV2 (component 2);
4) Ad26-CAG-S-CoV2 (component 1), Ad5-CMV-S-CoV2 (component 2);
5) Ad26-CAG-S-CoV2 (component 1), Ad5-CAG-S-CoV2 (component 2);
6) Ad26-CAG-S-CoV2 (component 1), Ad5-EF1-S-CoV2 (component 2);
7) Ad26-EF1-S-CoV2 (component 1), Ad5-CMV-S-CoV2 (component 2);
8) Ad26-EF1-S-CoV2 (component 1), Ad5-CAG-S-CoV2 (component 2);
9) Ad26-EF1-S-CoV2 (component 1), Ad5-EF1-S-CoV2 (component 2);
10) Ad26-null (component 1), Ad5-null (component 2);
11) Ad26-CMV-S-CoV2 (component 1), simAd25-CMV-S-CoV2 (component 2);
12) Ad26-CMV-S-CoV2 (component 1), simAd25-CAG-S-CoV2 (component 2);
13) Ad26-CMV-S-CoV2 (component 1), simAd25-EF1-S-CoV2 (component 2);
14) Ad26-CAG-S-CoV2 (component 1), simAd25-CMV-S-CoV2 (component 2);
15) Ad26-CAG-S-CoV2 (component 1), simAd25-CAG-S-CoV2 (component 2);
16) Ad26-CAG-S-CoV2 (component 1), simAd25-EF1-S-CoV2 (component 2);
17) Ad26-EF1-S-CoV2 (component 1), simAd25-CMV-S-CoV2 (component 2);
18) Ad26-EF1-S-CoV2 (component 1), simAd25-CAG-S-CoV2 (component 2);
19) Ad26-EF1-S-CoV2 (component 1), simAd25-EF1-S-CoV2 (component 2);
20) Ad26-null (component 1), simAd25-null (component 2);
21) simAd25-CMV-S-CoV2 (component 1), Ad5-CMV-S-CoV2 (component 2);
22) simAd25-CMV-S-CoV2 (component 1), Ad5-CAG-S-CoV2 (component 2);
23) simAd25-CMV-S-CoV2 (component 1), Ad5-EF1-S-CoV2 (component 2);
24) simAd25-CAG-S-CoV2 (component 1), Ad5-CMV-S-CoV2 (component 2);
25) simAd25-CAG-S-CoV2 (component 1), Ad5-CAG-S-CoV2 (component 2);
26) simAd25-CAG-S-CoV2 (component 1), Ad5-EF1-S-CoV2 (component 2);
27) simAd25-EF1-S-CoV2 (component 1), Ad5-CMV-S-CoV2 (component 2);
28) simAd25-EF1-S-CoV2 (component 1), Ad5-CAG-S-CoV2 (component 2);
29) simAd25-EF1-S-CoV2 (component 1), Ad5-EF1-S-CoV2 (component 2);
30) simAd25-null (component 1), Ad5-null (component 2);
31) phosphate-buffered saline

Three weeks later, blood samples were taken from the tail vein of the animals, and blood serum was separated. An enzyme-linked immunosorbent assay (ELISA) was used to measure antibody titers according to the following protocol:
1) Protein (S) was adsorbed onto wells of a 96-well ELISA plate for 16 hours at +4° C.
2) Then, for preventing a non-specific binding, the plate was “blocked” with 5% milk dissolved in TPBS in an amount of 100 μl per well. It was incubated in shaker at 37° C. for one hour.
3) Serum samples from the immunized mice were diluted using a 2-fold dilution method. Totally, 12 dilutions of each sample were prepared.
4) 50 μl of each of the diluted serum samples were added to the plate wells.
5) Then, incubation at 37° C. for 1 hour was performed.
6) After incubation the wells were washed three times with phosphate buffer.
7) Then, secondary antibodies against mouse immunoglobulins conjugated with horseradish peroxidase were added.
8) Next, incubation at 37° C. for 1 hour was performed.
9) After incubation the wells were washed three times with phosphate buffer.
10) Then, tetramethylbenzidine (TMB) solution was added which serves as a substrate for horseradish peroxidase and is converted into a colored compound by the reaction. The reaction was stopped after 15 minutes by adding sulfuric acid. Next, using a spectrophotometer, the optical density (OD) of the solution was measured in each well at a wavelength of 450 nm.

Antibody titer was determined as the last dilution at which the optical density of the solution was significantly higher than in the negative control group. The obtained results (geometric mean) are presented in Table 3.

TABLE 3
Antibody titers against S protein in the blood serum of mice
(geometric mean of antibody titers)

| | | Titer |
|---|---|---|
| No. | Name of animal group | of antibodies |
| 1 | Ad26-CMV-S-CoV2 (component 1), | 33,779 |
| | Ad5-CMV-S-CoV2 (component 2) | |
| 2 | Ad26-CMV-S-CoV2 (component 1), | 29,407 |
| | Ad5-CAG-S-CoV2 (component 2) | |
| 3 | Ad26-CMV-S-CoV2 (component 1), | 33,779 |
| | Ad5-EF1-S-CoV2 (component 2) | |
| 4 | Ad26-CAG-S-CoV2 (component 1), | 38,802 |
| | Ad5-CMV-S-CoV2 (component 2) | |
| 5 | Ad26-CAG-S-CoV2 (component 1), | 38,802 |
| | Ad5-CAG-S-CoV2 (component 2) | |
| 6 | Ad26-CAG-S-CoV2 (component 1), | 38,802 |
| | Ad5-EF1-S-CoV2 (component 2) | |
| 7 | Ad26-EF1-S-CoV2 (component 1), | 33,779 |
| | Ad5-CMV-S-CoV2 (component 2) | |
| 8 | Ad26-EF1-S-CoV2 (component 1), | 38,802 |
| | Ad5-CAG-S-CoV2 (component 2) | |
| 9 | Ad26-EF1-S-CoV2 (component 1), | 33,779 |
| | Ad5-EF1-S-CoV2 (component 2) | |
| 10 | Ad26-null (component 1), | 0 |
| | Ad5-null (component 2) | |
| 11 | Ad26-CMV-S-CoV2 (component 1), | 38,802 |
| | simAd25-CMV-S-CoV2 (component 2) | |
| 12 | Ad26-CMV-S-CoV2 (component 1), | 38,802 |
| | simAd25-CAG-S-CoV2 (component 2) | |
| 13 | Ad26-CMV-S-CoV2 (component 1), | 33,779 |
| | simAd25-EF1-S-CoV2 (component 2) | |
| 14 | Ad26-CAG-S-CoV2 (component 1), | 33,779 |
| | simAd25-CMV-S-CoV2 (component 2) | |
| 15 | Ad26-CAG-S-CoV2 (component 1), | 33,779 |
| | simAd25-CAG-S-CoV2 (component 2) | |
| 16 | Ad26-CAG-S-CoV2 (component 1), | 33,779 |
| | simAd25-EF1-S-CoV2 (component 2) | |
| 17 | Ad26-EF1-S-CoV2 (component 1), | 38,802 |
| | simAd25-CMV-S-CoV2 (component 2) | |
| 18 | Ad26-EF1-S-CoV2 (component 1), | 33,779 |
| | simAd25-CAG-S-CoV2 (component 2) | |
| 19 | Ad26-EF1-S-CoV2 (component 1), | 33,779 |
| | simAd25-EF1-S-CoV2 (component 2) | |
| 20 | Ad26-null (component 1), | 0 |
| | simAd25-null (component 2) | |
| 21 | Ad25-CMV-S-CoV2 (component 1), | 33,779 |
| | Ad5-CMV-S-CoV2 (component 2) | |
| 22 | simAd25-CMV-S-CoV2 (component 1), | 29,407 |
| | Ad5-CAG-S-CoV2 (component 2) | |
| 23 | simAd25-CMV-S-CoV2 (component 1), | 25,600 |
| | Ad5-EF1-S-CoV2 (component 2) | |
| 24 | simAd25-CAG-S-CoV2 (component 1), | 33,779 |
| | Ad5-CMV-S-CoV2 (component 2) | |
| 25 | simAd25-CAG-S-CoV2 (component 1), | 29,407 |
| | Ad5-CAG-S-CoV2 (component 2) | |
| 26 | simAd25-CAG-S-CoV2 (component 1), | 29,407 |
| | Ad5-EF1-S-CoV2 (component 2) | |
| 27 | simAd25-EF1-S-CoV2 (component 1), | 33,779 |
| | Ad5-CMV-S-CoV2 (component 2) | |
| 28 | simAd25-EF1-S-CoV2 (component 1), | 38,802 |
| | Ad5-CAG-S-CoV2 (component 2) | |
| 29 | simAd25-EF1-S-CoV2 (component 1), | 33,779 |
| | Ad5-EF1-S-CoV2 (component 2) | |
| 30 | simAd25-null (component 1), Ad5-null | 0 |
| | (component 2) | |
| 31 | phosphate-buffered saline | 0 |

As shown in the presented data, all variants of the pharmaceutical agent induce humoral immune response against SARS-CoV-2 glycoprotein.

### EXAMPLE 9

### Assessment of the Effectiveness of Immunization with the Developed Pharmaceutical Agent in Comparison with the Control Product Containing One Serotype of Recombinant Adenovirus

The aim of this experiment was to compare the antibody titers against the SARS-CoV-2 virus S protein in the blood serum of mice following their immunization with different variants of the developed pharmaceutical agent, containing 2 different serotypes of recombinant adenovirus, with the antibody titers against the SARS-CoV-2 virus S protein in the blood serum of mice immunized twice with the control product containing one serotype of recombinant adenovirus.

In this experiment BalB/c mice, 18 g, 35 pcs. were used.

The animals were immunized with a 2-week interval:
1) Ad26-CMV-S-CoV2 (component 1), Ad5-CMV-S-CoV2 (component 2), 5*10⁶ v.p.;
2) Ad26-CMV-S-CoV2 (component 1), simAd25-CMV-S-CoV2 (component 2), 5*10⁶ v.p.;
3) simAd25-CMV-S-CoV2 (component 1), Ad5-CMV-S-CoV2 (component 2), 5*10⁶ v.p.;
4) Ad26-CMV-S-CoV2, Ad26-CMV-S-CoV2, 5*10⁶ v.p.;
5) Ad5-CMV-S-CoV2, Ad5-CMV-S-CoV2, 5*10⁶ v.p.;
6) simAd25-CMV-S-CoV2, simAd25-CMV-S-CoV2, 5*10⁶ v.p.;
7) PBS.

One month later, the antibody titer against SARS-CoV-2 virus S antigen was determined using an enzyme-linked immunosorbent assay (ELISA). The experiment results are presented below in the Table.

TABLE 4
Antibody titer against SARS-CoV-2 virus S antigen
in the blood of mice one month following their immunization with
the developed pharmaceutical agent and control products.

| Group name | Antibody titer |
|---|---|
| Ad26-CMV-S-CoV2 (component 1), | 3,104 |
| Ad5-CMV-S-CoV2 (component 2) | |
| Ad26-CMV-S-CoV2 (component 1), | 2,702 |
| simAd25-CMV-S-CoV2 (component 2) | |
| simAd25-CMV-S-CoV2 (component 1), | 3104 |
| Ad5-CMV-S-CoV2 (component 2) | |
| Ad26-CMV-S-CoV2, Ad26-CMV-S-CoV2 | 588 |
| Ad5-CMV-S-CoV2, Ad5-CMV-S-CoV2 | 512 |
| simAd25-CMV-S-CoV2, simAd25-CMV-S-CoV2 | 446 |
| PBS | 0 |

The presented data show that the immunization of animals with the pharmaceutical agent has a potentiating effect on immune response. This effect is proven by a significantly higher antibody titer against SARS-CoV-2 virus S antigen in the blood serum of animals immunized by the pharmaceutical agent, containing two vector types, as compared with the sum of antibody titers in the groups immunized with a single vector type.

### EXAMPLE 10

### Assessment of the Effectiveness of Immunization with the Developed Pharmaceutical Agent Based on the Evaluation of the Percentage of Proliferating Lymphocytes

The level of cell-mediated immunity against the SARS-Cov2 virus was assessed by determining the number of proliferating CD4+ and CD8+ lymphocytes of mouse peripheral blood in the culture in vitro following the second re-stimulation of cells with recombinant RBD fragment of the coronavirus S protein. In order to determine the numbers of proliferating CD4+ and CD8+ lymphocytes, a method of staining lymphocytes with CFSE dye was used. This method is based on the ability of the fluorescent non-toxic CFSE dye to incorporate easily into the cells. Following cell stimulation with an antigen, lymphocytes begin to proliferate, and the dye from the parent cell is distributed uniformly between the daughter cells. The label concentration and, consequently, the fluorescence intensity in the daughter cells is decreased precisely twice. Therefore, dividing cells can be easily traced by the reducing fluorescence intensity.

C57BL/6 mice were used in the experiment. All animals were divided into 31 groups (3 animals per group) and injected intramuscularly with component 1 of the pharmaceutical agent at a dose 10⁸ viral particles/100 μl. Two weeks later component 2 was injected at a dose 10⁸ viral particles/100 μl.. Thus, the following groups of animals were formed:
1) Ad26-CMV-S-CoV2 (component 1), Ad5-CMV-S-CoV2 (component 2);
2) Ad26-CMV-S-CoV2 (component 1), Ad5-CAG-S-CoV2 (component 2);
3) Ad26-CMV-S-CoV2 (component 1), Ad5-EF1-S-CoV2 (component 2);
4) Ad26-CAG-S-CoV2 (component 1), Ad5-CMV-S-CoV2 (component 2);
5) Ad26-CAG-S-CoV2 (component 1), Ad5-CAG-S-CoV2 (component 2);
6) Ad26-CAG-S-CoV2 (component 1), Ad5-EF1-S-CoV2 (component 2);
7) Ad26-EF1-S-CoV2 (component 1), Ad5-CMV-S-CoV2 (component 2);
8) Ad26-EF1-S-CoV2 (component 1), Ad5-CAG-S-CoV2 (component 2);
9) Ad26-EF1-S-CoV2 (component 1), Ad5-EF1-S-CoV2 (component 2);
10) Ad26-null (component 1), Ad5-null (component 2);
11) Ad26-CMV-S-CoV2 (component 1), simAd25-CMV-S-CoV2 (component 2);
12) Ad26-CMV-S-CoV2 (component 1), simAd25-CAG-S-CoV2 (component 2);
13) Ad26-CMV-S-CoV2 (component 1), simAd25-EF1-S-CoV2 (component 2);
14) Ad26-CAG-S-CoV2 (component 1), simAd25-CMV-S-CoV2 (component 2);
15) Ad26-CAG-S-CoV2 (component 1), simAd25-CAG-S-CoV2 (component 2);
16) Ad26-CAG-S-CoV2 (component 1), simAd25-EF1-S-CoV2 (component 2);
17) Ad26-EF1-S-CoV2 (component 1), simAd25-CMV-S-CoV2 (component 2);
18) Ad26-EF1-S-CoV2 (component 1), simAd25-CAG-S-CoV2 (component 2);
19) Ad26-EF1-S-CoV2 (component 1), simAd25-EF1-S-CoV2 (component 2);
20) Ad26-null (component 1), simAd25-null (component 2);
21) simAd25-CMV-S-CoV2 (component 1), Ad5-CMV-S-CoV2 (component 2);
22) simAd25-CMV-S-CoV2 (component 1), Ad5-CAG-S-CoV2 (component 2);
23) simAd25-CMV-S-CoV2 (component 1), Ad5-EF1-S-CoV2 (component 2);
24) simAd25-CAG -S-CoV2 (component 1), Ad5-CMV-S-CoV2 (component 2);
25) simAd25-CAG -S-CoV2 (component 1), Ad5-CAG-S-CoV2 (component 2);
26) simAd25-CAG -S-CoV2 (component 1), Ad5-EF1-S-CoV2 (component 2);
27) simAd25-EF1-S-CoV2 (component 1), Ad5-CMV-S-CoV2 (component 2);
28) simAd25-EF1-S-CoV2 (component 1), Ad5-CAG-S-CoV2 (component 2);
29) simAd25-EF1-S-CoV2 (component 1), Ad5-EF1-S-CoV2 (component 2);
30) simAd25-null (component 1), Ad5-null (component 2);
31) phosphate buffered saline.

At day 8 of the experiment, the animals were euthanized. Lymphocytes were isolated from the spleen by Ficoll-Urografin density gradient centrifugation. Then, the isolated cells were stained with CFSE according to the technique (B. J. Quah et. al., Monitoring lymphocyte proliferation in vitro and in vivo with the intracellular fluorescent dye carboxyfluorescein diacetate succinimidyl ester, Nature Protocols, 2007, [symbol] 2(9), 2049-2056) and cultured in the presence of antigen (SARS-CV-2 virus S glycoprotein).

Then, the cells were analyzed using cytofluorometry. The obtained results are shown in FIG. 1, 2, 3, 4. Thus, it could be concluded that all variants of the developed pharmaceutical agent induce antigen-specific immune response (both CD4+ and CD8+).

### EXAMPLE 11

### Assessment of the Protective Potency of the Developed Pharmaceutical Agent Against COVID-19 in Laboratory Animals

Protective efficacy of the developed pharmaceutical agent against COVID-19 was assessed in Syrian golden hamsters with induced immunodeficiency, using a model of lethal infection caused by the SARS-CoV-2 virus.

The animals were divided in 31 groups (8 animals per group) and immunized twice: with component 1 (at a dose 10⁸ viral particles/animal) and component 2 (at a dose 10⁸ viral particles/animal) of the developed pharmaceutical agent with a 21-day interval.

Thus, the following animal groups were formed:
1) Ad26-CMV-S-CoV2 (component 1), Ad5-CMV-S-CoV2 (component 2);
2) Ad26-CMV-S-CoV2 (component 1), Ad5-CAG-S-CoV2 (component 2);
3) Ad26-CMV-S-CoV2 (component 1), Ad5-EF1-S-CoV2 (component 2);
4) Ad26-CAG-S-CoV2 (component 1), Ad5-CMV-S-CoV2 (component 2);
5) Ad26-CAG-S-CoV2 (component 1), Ad5-CAG-S-CoV2 (component 2);
6) Ad26-CAG-S-CoV2 (component 1), Ad5-EF1-S-CoV2 (component 2);
7) Ad26-EF1-S-CoV2 (component 1), Ad5-CMV-S-CoV2 (component 2);
8) Ad26-EF1-S-CoV2 (component 1), Ad5-CAG-S-CoV2 (component 2);
9) Ad26-EF1-S-CoV2 (component 1), Ad5-EF1-S-CoV2 (component 2);
10) Ad26-null (component 1), Ad5-null (component 2);
11) Ad26-CMV-S-CoV2 (component 1), simAd25-CMV-S-CoV2 (component 2);
12) Ad26-CMV-S-CoV2 (component 1), simAd25-CAG-S-CoV2 (component 2);
13) Ad26-CMV-S-CoV2 (component 1), simAd25-EF1-S-CoV2 (component 2);
14) Ad26-CAG-S-CoV2 (component 1), simAd25-CMV-S-CoV2 (component 2);
15) Ad26-CAG-S-CoV2 (component 1), simAd25-CAG-S-CoV2 (component 2)
16) Ad26-CAG-S-CoV2 (component 1), simAd25-EF1-S-CoV2 (component 2);
17) Ad26-EF1-S-CoV2 (component 1), simAd25-CMV-S-CoV2 (component 2);
18) Ad26-EF1-S-CoV2 (component 1), simAd25-CAG-S-CoV2 (component 2);
19) Ad26-EF1-S-CoV2 (component 1), simAd25-EF1-S-CoV2 (component 2);
20) Ad26-null (component 1), simAd25-null (component 2)
21) simAd25-CMV-S-CoV2 (component 1), Ad5-CMV-S-CoV2 (component 2);
22) simAd25-CMV-S-CoV2 (component 1), Ad5-CAG-S-CoV2 (component 2);
23) simAd25-CMV-S-CoV2 (component 1), Ad5-EF1-S-CoV2 (component 2);
24) simAd25-CAG-S-CoV2 (component 1), Ad5-CMV-S-CoV2 (component 2);
25) simAd25-CAG-S-CoV2 (component 1), Ad5-CAG-S-CoV2 (component 2);
26) simAd25-CAG-S-CoV2 (component 1), Ad5-EF1-S-CoV2 (component 2);
27) simAd25-EF1-S-CoV2 (component 1), Ad5-CMV-S-CoV2 (component 2);
28) simAd25-EF1-S-CoV2 (component 1), Ad5-CAG-S-CoV2 (component 2);
29) simAd25-EF1-S-CoV2 (component 1), Ad5-EF1-S-CoV2 (component 2);
30) simAd25-null (component 1), Ad5-null (component 2);
31) phosphate buffered saline.

Immunosuppressants were administered starting from day 7 after the booster immunization; at day 14 after the booster immunization the animals were challenged intranasally with the SARS-CoV-2 virus at a dose 10⁶ TCID50 per animal in an amount of 50 μl.

In the course of experiment, the body weight of non-vaccinated animals in the control group was decreasing dramatically after the challenge (at day 10 the average weight loss of 32% of baseline weight). At the same time, the body weight of animals immunized with all variants of the pharmaceutical agent during the first days after the challenge was declining slightly and then increased (at day 10 the average weight gain of 11% of baseline weight).

FIG. 4 illustrates the survival rate of animals after the challenge. The study results have demonstrated that the immunization with all variants of the pharmaceutical agent provided protection against the lethal infection caused by the SARS-CoV-2 virus for 100% of animals with induced immunodeficiency. In the non-vaccinated control group the lethality rate was 100%.

Thus, in the model of Syrian golden hamsters with induced immunodeficiency it was demonstrated that immunization with the developed pharmaceutical agent induced a protective immune response which ensured protection of 100% of animals against the lethal infection caused by the SARS-CoV-2 virus.

### EXAMPLE 12

### Toxicity Studies of the Developed Pharmaceutical Agent

Toxicity was assessed in sexually mature outbred male and female mice. The experimental study was performed with the pharmaceutical agent, variant 1 (component 1: Ad26-CMV-S-CoV2, component 2: Ad5-CMV-S-CoV2); the pharmaceutical agent, variant 2 (component 1: Ad26-CMV-S-CoV2, component 2: simAd25-CMV-S-CoV2); the pharmaceutical agent, variant 3 (componentl: simAd25-CMV-S-CoV2, component 2: Ad5-CMV-S-CoV2). Each of the pharmaceutical agent components was injected intramuscularly and intravenously in escalating doses: 10⁸ v. p.; 10⁹ v. p.; 10¹⁰ v. p.; and; 10¹¹ v. p.

Neither animal deaths, nor intoxication signs were reported during the experiment. It was found that the vaccine did not have impact on the body weight or the weight of internal organs of experimental animals. The structure of the mouse internal organs was not affected, as verified at the necropsy performed 14 days following the administration of the vector-based vaccine. No topical irritating effects were recorded in the performed experimental study.

Thus, the study findings demonstrate the absence of toxicity of the developed pharmaceutical agent.

### EXAMPLE 13

### Immunogenicity Study of the Developed Pharmaceutical Agent According to Variant 1 in Primates

The aim of this experimental study was to assess the level of humoral and T-cell mediated immunity in primates after their immunization with the developed pharmaceutical agent.

The evolution of humoral immune response was assessed by the increase in antibody titers against the SARS-CoV-2 virus S protein and virus-neutralizing antibody titers in the blood of primates. The evolution of cell-mediated immune response was assessed by determining the number of proliferating CD4+ [symbol] CD8+ T lymphocytes.

This study involved 17 rhesus macaque males with a body weight ranging between 2.0 and 2.2 kg. The animals were vaccinated with the developed pharmaceutical agent, variant 1 (component 1: Ad26-CMV-S-CoV2, component 2: Ad5-CMV-S-CoV2). They received component 1 at a human therapeutic dose 10¹¹ viral particles/dose, and 21 days later—component 2 at a human therapeutic dose 10¹¹ viral particles/dose.

Blood samples were taken from all animals prior to vaccination (day 0) and 7, 14 and 28 days following the vaccination. Titers of specific antibodies against the SARS-CoV-2 virus S protein RBD were measured in the blood serum of primates after their immunization with the developed pharmaceutical agent, using ELISA technique as follows:
SARS-CoV-2 virus RBD antigen at a concentration 100 ng/well was immobilized on the plates;
two-fold dilutions of primate blood serum were made in the blocking buffer (dilutions 1:50-1:51200), incubated in plate (strip) wells with immobilized RBD-antigen;
after washing, the formed Ag-Ab complex was detected with a horseradish peroxidase-labeled conjugate specific to Fc-fragment of monkey antibody IgG (Anti-MONKEY IgG (gamma chain) (GOAT) Antibody—617-101-012, ROCKLAND).
after washing, a chromogenic substrate was added to the formed complex; then, for stopping the enzymatic reaction a stop reagent was used.

The developed color (absorption) was recorded using spectrophotometer Multiskan FC (Thermo) with two wave lengths: main filter—450 nm, reference filter—620 nm.

IgG antibody titer against the SARS-CoV-2 virus S protein was defined as a serum dilution in which the value of optical density is twice higher than the value of optical density in the negative control (blood serum of the same primate prior to the agent administration) in the same dilution. The experiment results are shown on FIG. 5.

The findings demonstrate that the antibody titer against the SARS-CoV-2 virus increased in all animals immunized with the developed pharmaceutical agent. With that, the peak antibody titer was recorded one week after the injection of component 2 (at day 28 of the experiment).

The level of virus-neutralizing antibodies in the blood of rhesus macaques was determined in the neutralization reaction based on the suppression of negative colonies formed by the SARS-CoV-2 virus in a one-day monolayer of Vero C1008 cells under agar overlay medium. The neutralization reaction was designed as follows: constant dose of virus—serum dilutions.

The study included the immune sera received from primates prior to vaccination (day 0), and 7, 14 and 28 days following the vaccination; positive control sample (blood serum sample from a human convalescent where the specific antibodies against the SARS-CoV-2 virus are present); negative control specimen (fetal calf serum (FCS) where the specific antibodies against the SARS-CoV-2 virus are absent); and, culture of the SARS-CoV-2 virus.

Dilution 1:5 of the blood sera was used in the neutralization reaction. The working dilution of virus-containing suspension based on the SARS-CoV-2 virus (“antigen”) was prepared in Hanks' solution with 2% FCS and antibiotics (streptomycin sulfate and benzylpenicillin sodium salt), 100 U/ml each, using serial decimal dilution. Concentration of the SARS-CoV-2 virus in the prepared dilution amounted to 200 PFU·ml⁻¹.

To conduct the experiment, plastic vials with a working surface area of 25 cm², Cellstar®, were selected for the neutralization reaction with a daily monolayer of Vero C1008 cells. A mixture of equal volumes of serum and SARS-CoV-2 virus culture was incubated for 60 minutes at a temperature range between 36.5° C. and 37.5° C., and then added in an amount of 0.5 ml to the monolayer of Vero C1008 cells (the growth medium was preliminarily removed). After the antigen+antibody complex was adsorbed on the cells for 60 minutes at 36.5-37.5° C., the inoculate was decanted. Then, primary agar overlay designed for the SARS-CoV-2 virus was applied, and the monolayer was further incubated at a temperature range between 36.5° C. and 37.5° C. for 2 days.

Following 2 days the infected cell monolayer was stained with 0.1% solution of neutral red. For doing this, the secondary agar overlay was applied and incubation was performed for 24 hours at 36.5-37.5° C., and the number of negative colonies in the vials was counted. An antibody titer in the tested serum was defined as the highest dilution of the blood serum in which the determined suppression of negative colonies, formed by the SARS-CoV-2 virus, was at least by 50% more than in the negative control.

It was demonstrated that the level of virus-neutralizing antibodies above 1:5 at day 14 of the experiment was found in 17.6% of animals, while at day 28 of the experiment—in 100% of animals.

Thus, the findings demonstrate that the administration of the developed pharmaceutical agent induces humoral immune response to the SARS-CoV-2 virus in primates.

To assess T-cell mediated immune response, mononuclear cells were separated from the blood of primates by density gradient centrifugation in Ficoll solution prior to vaccination (day 0), and at days 7, 14 and 28 after vaccination.

The method is based on floating density gradient of the blood cells. Using density gradient centrifugation with polysaccharide Ficoll solution in water it is possible to separate the peripheral blood cells and isolate a mononuclear cell fraction (MF) which includes lymphocytes, subpopulation of monocytes, blast hemopoietic cells, and a fraction containing granulocytes and erythrocytes.

The MF density is lower than that of Ficoll and therefore after centrifugation it is layered above the Ficoll reagent.

The density of granulocytes and erythrocytes is higher than the gradient density, they pass through the gradient and migrate to the test-tube bottom layer (Boyum A. Separation of leukocytes from blood and bone marrow //Scand.J.Clin.Lab.Investig.—1968.-Vol.21—Supp1.97.p.1-9). Platelets, the smallest cells, remain in the blood serum without reaching the interface of “water/Ficoll” phases, when centrifugation at the appropriate speed is performed.

Following the mononuclear cell fraction isolation from the peripheral blood of primates, the cells were stained with fluorescent dye CFSE (Invivogen, USA) and placed in plate wells.

After seeding mononuclear cells in plate wells, the lymphocytes were re-stimulated in vitro by adding RBD fragment of the coronavirus S protein to the culture medium (final protein concentration—1 μg/ml). Intact cells without added antigen were used as a negative control. The percentage of proliferating cells was measured 72 hours following the antigen addition.

The experiment results are presented on FIGS. 6 and 7.

The experiment data demonstrate that the maximum level of T-cell mediated immunity induced in primates by the immunization with the developed pharmaceutical agent was recorded at day 28 after the immunization as assessed by mean arithmetic value of the percentage of proliferating CD4+ T lymphocytes and CD8+ T lymphocytes. This finding is associated with the second (boost) immunization performed at day 21 of the study (1.2% vs. 0.1% in the non-immunized group). In this case, proliferating CD4+ and CD8+ T lymphocytes are re-stimulated for proliferation, increasing the percentage of their presence in the vaccinated animal.

In summary, a conclusion can be made that the immunization of primates with the developed pharmaceutical agent used in the tested dose and immunization regimen induces significant (with a statistically significant difference from the values in the control group of non-immunized animals) humoral immune response characterized by an increase in antibody titer against the SARS-CoV-2 virus S protein and neutralizing antibody titer. It also induces T-cell mediated immunity including both CD4+ and CD8+ lymphocytes.

### EXAMPLE 14

The level of cell-mediated immunity was assessed by determining the number of proliferating CD4+ and CD8+ lymphocytes

Evaluation of the immunogenicity of the developed pharmaceutical agent by assessing cell-mediated immune response to the SARS-CoV-2 virus antigen in the blood of volunteers at different time periods after vaccination

The level of cell-mediated immunity was assessed in clinical trials of the developed pharmaceutical agent according to variant 1.

The trial involved 40 volunteers immunized with:
1) component 1 and 21 days later—with component 2 of a liquid formulation of the developed pharmaceutical agent, variant 1 (component 1: Ad26-CMV-S-CoV2, component 2: Ad5-CMV-S-CoV2), at a dose 1×10¹¹ viral particles (20 individuals).
2) component 1 and 21 days later—with component 2 of a lyophilized (freeze-dried) formulation of the developed pharmaceutical agent, variant 1 (component 1: Ad26-CMV-S-CoV2,component 2: Ad5-CMV-S-CoV2), at a dose 1×10¹¹ viral particles (20 individuals).

Blood samples were taken from volunteers at days 0 (prior to vaccination) 7, 14 and 28, and mononuclear cells were separated from the blood by density gradient centrifugation in Ficoll solution. Then, the isolated cells were stained with fluorescent dye CFSE (Invivogen, USA) and seeded in plate wells.

Next, lymphocytes were re-stimulated in vitro by adding the coronavirus S protein to the culture medium (final protein concentration—1 μg/ml). Intact cells without added antigen were used as a negative control. The percentage of proliferating cells was determined 72 hours after the antigen addition, and the culture medium was sampled for measuring gamma-interferon.

For determining % of proliferating cells, they were stained with the antibodies against marker molecules of T lymphocytes CD3, CD4, CD8 (anti-CD3 Pe-Cy7 (BD Biosciences, clone SK7), anti-CD4 APC (BD Biosciences, clone SK3), anti-CD8 PerCP-Cy5.5 (BD Biosciences, clone SK1)). Proliferating (cells with a lower amount of CFSE dye) CD4+ and CD8+ T lymphocytes were determined in the cell mixture, using high-performance cytofluorometer BD FACS AriaIII (BD Biosciences, USA).

The resulting percentage of proliferating cells in each specimen was determined by subtracting the result obtained in the analysis of intact cells from the result obtained in the analysis of cells re-stimulated by the coronavirus S antigen. The obtained results are shown on FIGS. 8 and 9 (for liquid formulation of the vaccine) and FIGS. 10 and 11 (for lyophilized formulation of the vaccine).

The quantitative measurement of gamma-interferon (IFN [symbol] ) concentration in the culture medium of mononuclear cells from the human blood 72 hours following their re-stimulation with the coronavirus S protein was performed using a “gamma-Interferon-IFA-BEST” (VECTOR-BEST, Russia) a kit according to the manufacturer's instruction. The received data are presented on FIG. 12 (for liquid formulation of the vaccine) and FIG. 13 (for lyophilized formulation of the vaccine).

The results of the performed study demonstrated that the level of cell-mediated immunity induced by the sequential immunization of volunteers with both formulations of the pharmaceutical agent, variant 1 (based on the median numbers of proliferating CD4+ and CD8+ T lymphocytes) was increasing as more days passed since the date of the immunization.

In both groups, the peak values of proliferating CD4+ and CD8+ T lymphocytes were recorded at day 28 after the immunization. The largest statistically significant difference in the values of proliferating CD4+ and CD8+ T lymphocytes (p<0.001) was reported between their values at day 0 and day 28 of the study.

Based on the results shown on FIGS. 12 and 13, a conclusion can be made that the level of cell-mediated immunity induced by the sequential immunization of volunteers with both formulations of the pharmaceutical agent, variant 1 (according to the median growth of IFNγ concentration) was increasing as more days passed since the date of the immunization.

A statistically significant difference in the values of increase in IFNγ concentration prior to immunization (day 0) and at 14 day after the vaccination was p<0.001. The maximum increase in IFNγ concentration was found on day 28 following the immunization. The largest statistically significant difference in the values of increase in IFNγ concentration (p<0.001) was reported between day 0 and day 28 of the study.

Thus, based on the findings a conclusion can be made that the immunization with the developed pharmaceutical agent is capable to induce the formation of strong antigen-specific cell-mediated anti-infection immunity which is confirmed by a high level of statistic significance in the measured parameters prior and following the immunization.

### EXAMPLE 15

### Evaluation of the Immunogenicity of the Developed Pharmaceutical Agent by Assessing Antibody Titer Against the SARS-CoV-2 Virus Antigen in the Blood of Volunteers at Different Time Periods after Vaccination

The trial involved 40 volunteers immunized with:
1) component 1, and 21 days later—with component 2 of liquid formulation of the developed pharmaceutical agent, variant 1 (component 1: Ad26-CMV-S-CoV2, component 2: Ad5-CMV-S-CoV2), at a dose 1×10¹¹ viral particles (20 individuals).
2) component 1 and 21 days later—with component 2 of lyophilized (freeze-dried) formulation of the developed pharmaceutical agent, variant 1 (component 1: Ad26-CMV-S-CoV2, component 2: Ad5-CMV-S-CoV2), at a dose 1×10¹¹ viral particles (20 individuals).

Blood samples were taken from volunteers at days 7, 14 and 28, and the serum was separated from the blood.

Antibody titer against the SARS-CoV-2 virus S protein RBD was measured using an enzyme-linked immunosorbent assay (ELISA) with a test kit “SARS-CoV-2-RBD-IFA-Gamaleya.” The assay was performed in accordance with the manufacturer's instruction.

The resulting assay measurements of antibody titer against SARS-CoV-2 virus antigen in the blood serum of volunteers after receiving liquid formulation of the product are shown on FIG. 14.

The resulting assay measurements of antibody titer against the SARS-CoV-2 virus antigen in the blood serum of volunteers after receiving lyophilized (freeze-dried) formulation of the product are shown on FIG. 15.

As demonstrated by the findings, the immunization of volunteers with the developed pharmaceutical agent, both as a liquid and lyophilized (freeze-dried) formulation helped to achieve a strong (with a statistically significant difference from the values in control, non-immunized group of volunteers) humoral immunity characterized by an increase in antibody titer against the SARS-CoV-2 virus S protein. With that, the level of humoral immune response was growing as more days have passed since the date of immunization.

Thus, the assigned technical aim, in particular, the development of agents ensuring the effective induction of immune response against the SARS-CoV-2 virus is accomplished as proven by the provided examples.

### INDUSTRIAL APPLICABILITY

All the provided examples confirm the effectiveness of the pharmaceutical agents ensuring the effective induction of immune response against the SARS-CoV-2 virus and the industrial applicability.

1. A pharmaceutical agent for the induction of specific immunity against severe acute respiratory syndrome virus (SARS-CoV-2), comprising:
component 1, comprising:
an expression vector composition, comprising:
a genome of recombinant human adenovirus serotype 26, wherein the E1 and E3 regions are deleted from the genome and the ORF6-Ad26 region is replaced by ORF6-Ad5 with an expression cassette selected from SEQ ID NO:1, SEQ ID NO:2, or SEQ ID NO:3; and component 2, comprising:
an expression vector composition, comprising:
a genome of recombinant human adenovirus serotype 5, wherein the E1 and E3 regions are deleted from the genome with an expression cassette selected from SEQ ID NO:1, SEQ ID NO:2, or SEQ ID NO:3.

2. A pharmaceutical agent for the induction of specific immunity against severe acute respiratory syndrome virus (SARS-CoV-2), comprising:
component 1, comprising:
an expression vector composition, comprising:
a genome of recombinant human adenovirus serotype 26, wherein the E1 and E3 regions are deleted from the genome and the ORF6-Ad26 region is replaced by ORF6-Ad5 with an expression cassette selected from SEQ ID NO:1, SEQ ID NO:2, or SEQ ID NO:3; and
component 2, comprising:
an expression vector composition, comprising:
a genome of recombinant simian adenovirus serotype 25, wherein the E1 and E3 regions are deleted from the genome with an expression cassette selected from SEQ ID NO:4, SEQ ID NO:2, or SEQ ID NO:3.

3. A pharmaceutical agent for the induction of specific immunity against severe acute respiratory syndrome virus (SARS-CoV-2), comprising:
component 1, comprising:
an expression vector composition, comprising:
a genome of recombinant simian adenovirus serotype 25, wherein the E1 and E3 regions are deleted from the genome with an expression cassette selected from SEQ ID NO:4, SEQ ID NO:2, or SEQ ID NO:3: and
component 2, comprising:
an expression vector composition, comprising:
a genome of recombinant human adenovirus serotype 5, wherein the E1 and E3 regions are deleted from the genome with an expression cassette selected from SEQ ID NO:1, SEQ ID NO:2, or SEQ ID NO:3.

4. The pharmaceutical agent of claim 1, manufactured as a liquid or lyophilized (freeze-dried) formulation.

5. The pharmaceutical agent of claim 4, wherein the buffer solution for liquid formulation contains, by mass %:

| | |
|---|---|
| tris | from 0.1831 to 0.3432 |
| sodium chloride | from 0.3313 to 0.6212 |
| sucrose | from 3.7821 to 7.0915 |
| magnesium chloride | from 0.0154 to 0.0289 |
| hexahydrate | |
| EDTA | from 0.0029 to 0.0054 |
| Polysorbate-80 | from 0.0378 to 0.0709 |
| ethanol 95% | from 0.0004 to 0.0007 |
| water | The remaining part. |

6. The pharmaceutical agent of claim 4, wherein the buffer solution for lyophilized (freeze-dried) formulation contains, by mass %:

| | |
|---|---|
| tris | from 0.0180 to 0.0338 |
| sodium chloride | from 0.1044 to 0.1957 |
| sucrose | from 5.4688 to 10.2539 |
| magnesium chloride | from 0.0015 to 0.0028 |
| hexahydrate | |
| EDTA | from 0.0003 to 0.0005 |
| Polysorbate-80 | from 0.0037 to 0.0070 |
| water | the remaining part. |

7. The pharmaceutical agent of claim 1, wherein component 1 and component 2 are placed in different packages.

8. A method of inducing specific immunity against severe acute respiratory syndrome virus (SARS-CoV-2), comprising:
using the pharmaceutical agent of claim 1, wherein component 1 and component 2 are used in an effective amount, sequentially, with a time interval of no less than one week.

9. The pharmaceutical agent of claim 2, manufactured as a liquid or lyophilized (freeze-dried) formulation.

10. The pharmaceutical agent of claim 3, manufactured as a liquid or lyophilized (freeze-dried) formulation.

11. The pharmaceutical agent of claim 2, wherein component 1 and component 2 are placed in different packages.

12. The pharmaceutical agent of claim 3, wherein component 1 and component 2 are placed in different packages.

13. A method of inducing specific immunity against severe acute respiratory syndrome virus (SARS-CoV-2), comprising:
using the pharmaceutical agent of claim 2, wherein component 1 and component 2 are used in an effective amount, sequentially, with a time interval of no less than one week.

14. A method of inducing specific immunity against severe acute respiratory syndrome virus (SARS-CoV-2), comprising:
using the pharmaceutical agent of claim 3, wherein component 1 and component 2 are used in an effective amount, sequentially, with a time interval of no less than one week.


SEQUENCE LISTING (7 sequences)

SEQ ID NO: 1 - DNA, 4711 bp, Artificial Sequence
  Synthetic Developed expression cassette, containing the CMV-promoter, optimized SARS-CoV-2 S protein sequence and polyadenylation signal
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 2 - DNA, 5984 bp, Artificial Sequence
  Synthetic Developed expression cassette, containing the CAG-promoter, optimized SARS-CoV-2 S protein sequence and polyadenylation signal
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctccccccctcccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcgggggggctgcgaggggaacaaaggctgcgtgcggggtgtgtgcgtggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcggggggacggggcagggcggggttcggcttctggcgtgtgaccggcggctctagaaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagatttttttatattttgttttgtgttattttttctttaacatccctaaaattttccttacatgttttactagccagatttttcctcctctcctgactactcccagtcatagctgtccctcttctcttatggagatc

SEQ ID NO: 3 - DNA, 5314 bp, Artificial Sequence
  Synthetic Developed expression cassette, containing the EF1-promoter, optimized SARS-CoV-2 S protein sequence and polyadenylation signal
ggtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcacaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatctagagtcggggcggccggccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccgagtgtcgataag

SEQ ID NO: 4 - DNA, 4678 bp, Artificial Sequence
  Synthetic Developed expression cassette, containing the CMV-promoter, optimized SARS-CoV-2 S protein sequence and polyadenylation signal
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatcgcggccgctcgagtctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 5 - DNA, 33765 bp, Artificial Sequence
  Synthetic Parental sequence of recombinant human adenovirus serotype 26, wherein the E1 and E3 regions are deleted, and ORF6-Ad26 is replaced by ORF6-Ad5
catcatcaataatataccccacaaagtaaacaaaagttaatatgcaaatgagcttttgaattttaacggttttggggcggagccaacgctgattggacgagaaacggtgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtgaaattaggtcattttggcgcgaaaactgaatgaggaagtgaaaagcgaaaaataccggtccctcccagggcggaatatttaccgagggccgagagactttgaccgattacgtgggggtttcgattgcggtgtttttttcgcgaatttccgcgtccgtgtcaaagtccggtgtttatgtcacagatcagctggtttcctttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcggatatcttatgtgtaatgtaatttgactcctttgagcacgggctcagaatcgtcctcgtcgaacttgcagcagctgccacagctacaacagcccttcaggcagctacagcagctggtcatgcaacacagcatgattgtgaccatcacgatggcaatcagtccggcgataaagcccagccagatgtaccagggccacttgatgtactgctcgtacttccccagttcttgcaggtcgatcaggctctcgttcagattcttggccacctcgttcagccggtcgatctctttctggatgttcacgacgctggcattgattccgctgatgtcgcccaggtccacgtcggggcttgtgtggttcttaaagtacttatccagttcctctttgaagctgtccagctcgggctgcagagggtcgtacacggtattgttcacaatgccgatcacgacgtcgcagttgccagacacgaaggtgttgtcggtggtgatgatctggggctcgtagaagttccgctgggtcacgaaccaatgggtgccgttggacacgaacacgccttctctaggaaagtgggctttgccgtcgtggcagatggctggagcggtggtgaaattcttctcttgagcgggcacgtatgtcacgtgcagaaacaccacgccgtgaggggcagactgagggaagctcatcaggtggtagcccttgccgcaaaagtccactctcttgctctggcccagcacacactcagacatcttggtggcggccagattggcagaggctctaatctcggcggctctgatcagctgctgggtaacgtaggtctgcagggactgcagccttccggtgatcagtctgtcgatctgcacctcggcttccaccttgtccagtctgctcaggatgtcgttcagcacagagctgatggcgccgaagttggaggacagctgcttgaccagggtgttcagtgcctgggcattctggttgaccacgtcctgcagctttcccagggcgcttgctgtgctgctcaggctgtcctggatcttgccgatggcgctgttgaactggttggcgatcagcttctggttctcgtacagcacattctgggtcactccgatgccgttgaaccggtaggccatctgcatagcaaaggggatctgcagagcggcgccagctccaaatgtccagccgcttgtgattgtgccggccagcagggcagatgtgtactgggcgatcatctcatcggtcagcagtggtggcagcactgtcagtccgttaaacttctgggcgcaaatcagatccctggcggcaatgtcgcccagacaatcgccatactgcttgatgaagccggcgtcggccagtgtcactttgttgaacagcaggtcctcgatgaagctccgcttgctgggcttgctaggatcgggcagaatctggctgaaattgaagccgccgaagtccttgataggaggggtcttgtagatctgcttcacttgggcgaacacctcttgggtgttcttgtcctgttccacggcgatccctgtcagggctctattcagctgggtgcagaagctgccgtactgcagcagcaggttggagcactcggtggaatcgccgcagatgtacatggtgcagtccacgctggtcttggtcatggacacaggcaggatctctgtggtcacgctgatggtgaagttggtggggatagcgatagagttgttggagtaggccacgctgttctcggcgcccagagacattgtgtaggcaatgatgctctggctggccacagatctggcccgtctggggctgtttgtctgtgtctggtagctggcacagatgccagcgccgatggggatgtcgcactcgtagctattgttcacgtgctcggctccgatcagacagccggctctggtctgaaacacattgctgccggtggagtacacccgccatgtaggtgtcagctgatcggcgtgaatggccacgggcacttcggtacagttcacgtcctggtacagcactgccacctgattgctggtgttggtgccaggggtgatcacagacactccgccgaagctgcaaggggtgatgtccaggatttccagtgtctggggatctcgtacggcgtctgtggtatcggcaatatcccggccaaactgctggaatggcaggaacttcttgttgctctctgtcagcacgccggtgccggtcaggccgttgaagttgaagttcacgcatttgttcttcacgagattggtgcttttcttagggccgcacactgtggcaggggcatgcagcagttcgaagctcagcaccaccactctgtagggctgatagcccacgccatttgtgggctgaaagccgtaggactgcagtgggaagtagcagttgaagccttccacgccgttacaaggggtgctgccggcctgatagatctcggtggagatgtcccgctcgaagggcttcagattggacttccggaacagccggtacaggtaattgtagttgccgccgactttggagtccaggttgttgctgttccaggcaatcacacagccggtgaagtcgtcgggcagcttgtagttgtagtcggcgatcttgcctgtctgtccaggggcaatctgccgcacttcatctccccggatcacgaagctgtcggcgtacacgtttgtgaagcacaggtcgttcagcttggtaggggacacgccgtagcacttgaaggtgctgaagctggcggagttgtacagcacggagtagtcggccacgcaattgctgatccgcttccggttccaggcgtacacagaggcgaatctggtggcattgaacacctcgccgaaggggcacagattggtgatattggggaaccgcacgatggattcggtgggctgcacccggaagttgctggtctggtagatgcccttttccacggtgaaggacttcagggtgcactttgtctcgctcagaggatccagagcacaatccacggcgtcggtgatggtgccgttctcgttgtacttcagcaggaaggttctaggctgcaggtagcccacatagtaagcggcggcaccagctgtccatccgctgctgctatcgccaggtgtcaggtagcttctgtgcagggccagcagtgtctgaaaccgggtgatgttgatgccgatgggcagatccaccaggggttccagagcagagaagccctgaggcagatcccgcacgaggttgataggggtgtgcttgctgtagatcttgaagtagccgtcgatgttcttgaacacgaactcgcgcaggttcttgaagttgccctgcttgccttccaggtccatcaggaaaggctgggacacgtactcgaaggtgcagttgttggcgctgctgtacacccggaactcgctttccatccagctcttgttgttcttgtgatagtagacgcccaggaaggggtcgttgcagaactggaactcgcacactttgatgaccacgttggtggcgttgttcacgatcagcaggctctgggtcttgctgtccagtgtggtgccgaagatccagcctctgatgatgttggacttctcggtgctggcaaagtacaccccgtcgttgaagggcagcacggggttgtcgaatctcttggtgccattggtgccggacacgtggatggcgtggaaccaggtcacgttgctgaagaaaggcaggaacaggtcctgggtagagtgcagcacgctggatctgaacaccttgtcggggtagtacacgcctctggtaaagctgttggtgtaggctggaggcagctgggttcttgtggtcaggttcacacactgactagagactagtggcaataacacaaggaacacaaacatggtaccaagcttaggctcgagcggccgcgtcgacggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaacagtgtttaaacgtatacctataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggaactcgtcgctcgacagcaccgccgcagccgcggcagccgcagccgccatgacagcgacgagactggcctcgagctacatgcccagcagcggtagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgtgggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcaaaacggttgagctgggagggatgcatgcggggggagatgatgtgcagtttggcctggatcttgaggttggcgatgttgccacccagatcccgccgggggttcatgttgtgcaggaccaccagaacggtgtagcccgtgcacttggggaacttgtcatgcaacttggaagggaatgcgtggaagaatttggagacgcccttgtgcccgcccaggttttccatgcactcatccatgatgatggcaatgggcccgtgggctgcggctttggcaaagacgtttctggggtcagagacatcgtaattatgctcctgggtgagatcatcataagacattttaatgaatttggggcggagggtgccagattgggggacgatggttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcttgtctctgacttggaggttttcccggacgagctcgccgaggaggcggtccccgcccagcgagagaagctcttgcagggaagcaaagtttttcaggggcttgagcccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccgcgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaggcgtccgctccgcagtgggcgcagacggtctcgcactcgactagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcagaccactctgagacgaaggcgcgcgtccacgccaagacaaaggaggccacgtgcgaggggtagcggtcgttgtccaccagggggtccaccttttccacggtatgcaggcacatgtccccctcctccgcatccaagaaggtgattggcttgtaggtgtaggccacgtgacctggggttcccgacgggggggtataaaagggggcgggtctgtgctcgtcctcactctcttccgcgtcgctgtccacgagcgccagctgttggggtaggtattccctctcaagagcgggcatgacctcggcactcaggttgtcagtttctagaaacgaggaggatttgatgtgggcctgccctgccgcgatgctttttaggagactttcatccatctggtcagaaaagactatttttttattgtcaagcttggtggcgaaggagccatagagggcgtttgagagaagcttggcgatggatctcatggtctgatttttgtcacggtcggcgcgctccttggccgcgatgttgagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcgtccaaggccatctgccactcgcgggcggccagcgctcgctcgtaggggttgaggggcggaccccaaggcatgggatgcgtgagggcggaggcgtacatgccgcagatgtcatagacatagatgggctccgagaggatgccgatgtaggtgggatagcagcgccccccgcggatgcttgcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtgaggcaggcggaccgagtcgcggatgaagtgcgcgtaggagtcttgcagcttggcgacgagctcggcggtgacgaggacgtccatggcgcagtagtccagcgtttcgcggatgatgtcataactcgcctctcctttcttctcccacagctcgcggttgagggcgtattcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcaaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcaaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaaacgcacggcgagatcgcgagcgagggcgaccagctctgggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctctgcgtcattgacggcggcctgacgcaggatctcttgcacgtcgcccgagttgtcctggtaggcgatctcggacatgaactgttcgatctcctcctcctggagatcgccgcggcccgcgcgctccacggtggcggcgaggtcattggagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcaaagacggcgtagttgcgcaggcgctggaagaggtagttgagggtggtggcgatgtgctcggtgacgaagaagtacatgatccagcggcgcaggggcatctcgctgatgtcgccgatggcttccagcctttccatggcctcgtagaagtccacggcgaagttgaaaaactgggcgttgcgggccgagaccgtgagctcgtcttccaggagccggatgagttcggcgatggtggcgcgcacctcgcgctcgaaatccccgggggcctcctcctcttcctcttcttccatgacgacctcttcttctatttcttcctctgggggcggtggtggtggcgggggccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccattgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgcggacgctgttagaattgcggttgctgatgatgtaattgaagtaggcgtttttgaggcggcggatggtggcgaggaggaccaggtccttgggtccagcttgctggatgcggagccgctcggccatgccccaggcctggccctgacaccggctcaggttcttgtagtagtcatgcatgagcctctcaatgtcatcactggctgaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtgataggccccggtgttgatggtgtaggtgcagttggccatgagcgaccagttgacggtctgcaggcctggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgctggccgaggggggtcgctagacttgaaagcgaccgaaaaccctgccgggtagtggctcgcgcccgtagtctggagaagcatcgccagggttgagtcgcggcagaacccggttcgaggacggccgcggcgagcgggacttggtcaccccgccgatataaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccaccacagacagagatggacttggaagagggcgaagggctggcaagactgggggcgccgtccccggagcgacatccccgcgtgcagctgcagaaggacgtgcgcccggcgtacgtgcctacgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcacgcgcgcacgtggcggcagccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgattaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcacgagccggcggcgcgagctaagcgaccgcgagctgatgctgagtctgcgccgggcgctggtagggggcgccgccggcggcgaggagtcctacttcgacatgggtgcggacctgcattggcagccgagccggcgcgccttggaggccgcctacggttcagaggacttggatgaggaagaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggatcggctggtgacggacgtgcgcgaggccgtggcgcagcgcgagcggttcaagaacgagggcctgggctcgctggtggcgctgaacgccttcctggcaacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaatctgcgcgggctgtggggcgtgcaggcgcccgtgggcgaccggtcaacggtgagcagcttgctgacgcccaactcgcggctgctgctgctgctgatcgcgcccttcaccgacagcggcagcgtgaaccgcaactcgtacctgggccatctgctgacgctgtaccgcgaggccataggccaggcgcaggtggacgagcagaccttccaggagatcactagcgtgagccgcgcgctggggcagaacgacaccgacagtctgagggccaccctgaactttttgctgaccaatagacagcagaagatcccggcgcagtacgcactgtcggccgaggaggaaaggattctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacttgcaccgcgcggcggccatgaacacggactactttaccaacgccatcctgaacccgcactggctcccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggccaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacactaacatgcccaatgtgaacgagtacatgttcaccagcaagtttaaggcgcgggtgatggtgtctaggaagcatccagagggggtagttgaaacagatttgagtcaggataagcttgaatatgagtggtttgagtttaccctgcccgagggaaacttttccgagaccatgaccatagacctgatgaacaacgccatcttggaaaactacttgcaagtggggcggcagaatggcgtgctggagagcgatatcggagtcaagtttgacagcagaaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaggatctagaaggtggcaacatccccgccctccttgatgtgcccaagtacttggaaagcaagaagaaagttgaagacgaaactaaaaatgcagctgcggccacagccgatacaaccactaggggtgatacatttgcaactccagcgcaagagacagcagctgataagaaggtagaagtcttgcccattgaaaaggatgagagtggtagaagttacaacctgatccaggggacccacgacacgctgtaccgcagttggtacctgtcctatacctacggggaccccgagaagggggtgcagtcgtggacgctgctcaccaccccggacgttacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaaatgtctattctcatctcgcccagcaataacaccggctggggtcttactagacccagcaccatgtacggaggagccaagaagcgctcccagcagcaccccgtccgcgtccgcggccacttccgcgctccctggggcgcttacaagcgcgggcggacttccaccgccgtgcgcaccaccgtcgacgacgtcatcgactcggtggtcgccgacgcgcgcaactacactcccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgccgccgccgctgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgtctccacttctaccgccgccacggctaccgagcctcccaggaggcgaagatggggcgccgccagccggctgatgcccaactacgtgttgcatccttccatcatcccgacgccgggctaccgcggcacccggtactacgccagccgccggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatccccataatcgccgcggccattggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccaaaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgaaaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccgcgcccgctccaccgaaggcaactccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccaccaccgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctatacgccactttcttccccatggcgcacaacaccgcttcaaccttggaagccatgctgcgcaacgacaccaacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaagcatgagcggctccagcgaacgagagctcgcggccatcgtgcgcgacctgggctgcgggccctactttttgggcacccacgacaagcgcttcccgggctttctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccactcagaccgtgcaggggcccgactccgccgcctgcggactcttctgttgcatgttcttgcatgccttcgtgcactggcccgaccgacccatggacggaaaccccaccatgaacttgctgacgggggtgcccaacggcatgctacaatcgccacaggtgctgcccaccctcaggcgcaaccaggaggaactctaccgcttcctcgcgcgccactccccttactttcgctcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggctgccacttgaactcggggatcaccagtttgggcactggggtctcggggaaggtctcgctccacatgcgccggctcatctgcagggcgcccagcatgtccggggcggagatcttgaaatcgcagttggggccggtgctctgcgcgcgcgagttgcggtacacggggttgcagcactggaacaccatcagactggggtacttcacactagccagcacgctcttgtcgctgatctgatccttgtccagatcctcggcgttgctcaggccgaacggggtcatcttgcacagctggcgtcccaggaagggcacgctctgaggcttgtggttacactcgcagtgcacgggcatcagcatcatccccgcgccgcgctgcatattcgggtagagggccttgacaaaggccgcgatctgcttgaaagcttgctgggccttggccccctcgctgaaaaacaggccgcagctcttcccgctgaactggttattcccacacccggcatcctgcacgcagcagcgcgcgtcatggctggtcagttgcaccacgctccgtccccagcggttctgggtcaccttagccttgctgggctgctccttcaacgcgcgctgcccgttctcgctggtcacatccatctccaccacgtggtccttgtggatcatcatcgtcccgtgcagacacttgagctggccttccacctcggtgcagccgtgatcccacagggcgcaaccggtgcactcccagttcttgtgcgcaatcccgctgtggctgaagatgtaaccttgcaacatgcggcccatgatggtgctaaatgctttctgggtggtgaaggtcagttgcatcccgcgggcctcctcgttcatccaggtctggcacatcttctggaagatctcggtctgctcgggcatgagcttgtaagcatcgcgcaggccgctgtcgacgcggtagcgttccatcagcacgttcatggtatccatgcccttctcccaggacgagaccagaggcagactcagagggttgcgtacgttcaggacaccgggggtcgcgggctcgacgatgcgttttccgtccttgccttccttcaatagaaccggcggctggctgaatcccactcccacgatcacggcatcttcctggggcatctcttcgtcggggtctaccttggtcacatgcttggtctttctggcttgcttcttttttggagggctgtccacggggagcacgtcctcctcggaagacccggagcccacccgctgatactttcggcgcttggtgggcagaggaggtggcggcgaggggctcctctcctgctccggcggatagcgcgccgacccgtggccccggggcggagtggcctctcggcccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtaccccccaaacgccagcccaacggcacctgcgagcccaacccgcgtctcaacttctatcccgtctttgcggtccccgaagccctcgccacctatcacatctttttcaagaaccaaaagatccccgtctcctgccgcgccaaccgcaccagcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagatgctcgcgcgctggctcgggacccacgacccccaggctttggaacagcggcgcaagctcatgctggccgtggtcctggttaccctcgagctggaatgcatgcgccgcttcttcagcgaccccgagaccctgcgcaaggtcgaggagaccctgcactacactttcagacacggtttcgtcaggcaggcctgcaagatctccaacgtggagctgaccaacctggtctcctgcctggggatcctgcacgagaaccgcctggggcagaccgtgctccactctaccctgaagggcgaggcgcggcgggactatgtccgcgactgcgtctttctatttctttgccacacatggcaagcagccatgggcgtgtggcaacagtgtctcgaggacgataacctgaaggagctggacaagcttcttgctagaaatcttaaaaagctgtggacgggcttcgacgagcgcaccgtcgcctcggacctggccgagatcgtgttccccgagcgcctgaggcagacgctgaaaggcgggctgcccgacttcatgagccagagcatgttgcaaaactaccgcactttcattctcgagcgatctgggatgctgcccgccacctgcaacgctttcccctccgactttgtcccgctgagctaccgcgagtgtcccccgccgctgtggagccactgctacctcttgcagctggccaactacatcgcctaccactcggacgtgatcgaggacgtgagcggcgaggggctgctcgagtgccactgccgctgcaacctgtgctccccgcaccgctccctggtctgcaacccccagctactaagcgagacccaggtcatcggtacctttgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgtccgcagcacgcggatctcacggcctgcgtcatcacccagggcgcaatcctcgcccaattgcacgccatccaaaaatcccgccaagagtttcttctgaaaaagggtagaggggtctacctggacccccagacgggcgaagtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccgccggtcacggatacaacctccgcagctccggccaagcctcctcgtagatgggatcgagtgaagggtgacggtaagcacgagcggcagggctaccgatcatggagggcccacaaagccgcgatcatcgcctgcttgcaagactgcggggggaacatcgctttcgcccgccgctacctgctcttccaccgcggggtaaacatcccccgcaacgtgttgcattactaccgtcaccttcacagctaagaaaaagcaagtaaaaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccaaatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagtttgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtccaaccccgaagttgcatgagaatttttgttgtactctttgtggtgagtttaataaaagctaaactcttgcaatactctggaccttgtcgtcgtcaactcaacgagaccgtctacctcaccaaccagactgaggtaaaactcacctgcagaccacacaagacctatatcatctggttcttcgagaacacctcatttgcagtctccaacactcactgcaacgacggtgttgaacttcccaacaacctttccagtggactgagttacgatacacatagagctaagctcgtcctctacaatccttttgtagagggaacctaccagtgccagagcggaccttgtactcacaccttccatttggtgaacgtcaccagcagcagcaacagctcagaaactaaccttccttctgatactaacaaacctcgtttcggaggtgagctaaggcttcccccttctgaggagggggttagcccatacgaagtggtcgggtatttgattttaggggtggtcctgggtgggtgcatagcggtgctagtcgagatggacggccaggcctccgagcagcgcatcctgcaactgcgcgtccgtcagcagcaggagcgtgccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctgctcaagaccctttgcggactccgcgacctcctccccatgaactgatgttgattaaaagcccaaaaaccaatcagccccttccccctaatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatggcaaagaggctccgggtggaagatgacttcaaccccgtctacccctatggctacgcgcggaatcagaatatccccttcctcactcccccctttgtctcctccgatggattcaaaaacttcccccctggggtcctgtcacttaaactggctgatccaatcaccatcaacaatggggatgtctcacttaaggtgggagggggacttgctgtagagcaacagactggtaacctaagcgtaaaccctgatgcacccttgcaagttgcaagtgataagctacagcttgctctggctcctccattcgaggtcagagatggaaagcttgctttaaaggcaggtaatggattaaaagtactagataattccattactggattgactggattattgaatacacttgtggtattaactggaaggggaataggaacggaggaattaaaaaatgacgatggtgtaacaaacaaaggagtcggcttgcgtgtaagacttggagatgacggcgggctgacatttgataaaaagggtgatttagtagcctggaataaaaaagatgacaggcgcaccctgtggacaacccctgacacatctccaaattgcaaaatgagtacagaaaaggattctaaacttacgttgacacttacaaagtgtggaagtcaggttctgggaaatgtatctttacttgcagttacaggtgaatatcatcaaatgactgctactacaaagaaggatgtaaaaatatctttactatttgatgagaatggaattctattaccatcttcgtcccttagcaaagattattggaattacagaagtgatgattctattgtatctcaaaaatataataatgcagttccattcatgccaaacctgacagcttatccaaaaccaagcgctcaaaatgcaaaaaactattcaagaactaaaatcataagtaatgtctacttaggtgctcttacctaccaacctgtaattatcactattgcatttaatcaggaaactgaaaatggatgtgcttattctataacatttaccttcacttggcaaaaagactattctgcccaacagtttgatgttacatcttttaccttctcatatcttacccaagagaacaaagacaaagactaataaaatgttttgaactgaatttatgaatctttatttatttttacaccagcacgggtagtcagtttcccaccaccagcccatttcacagtgtaaacaattctctcagcacgggtggccttaaacaggtcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaggtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaaggagctcgactgttcctcggtggacattgaaatggattctcttgcgtaccttgtcgtacttctgccagcagaaagtggctcgggaacagcagatacctttcctcctgctgtccttccgctgctgacgctcagtcatccaactgaagtacagccattcccgcaggttctccagcagctcctgtgcatctgatgaaacaaaagtcccgtcgatgcggattccccttaaaacatcagccaggacattgtaggccatcccaatccagttaatgcatcctgatctatcatgaagaggaggtgggggaagaactggaagaaccatttttattccaagcggtctcgaaggacgataaagtgcaagtcacgcaggtgacagcgttccccgccgctgtgctggtggaaacagacagccaggtcaaaacccactctattttcaaggtgctcgactgtggcttcgagcagtggctctacgcgtacatccagcataagaatcacattaaaggctggacctccatcgatttcatcaatcatcaggttacactcattcaccatccccaggtaattctcatttttccagccttggattatttctacaaattgttggtgtaagtccactccgcacatgtggaaaagttcccacagcgccccctccactttcataatcaggcagaccttcatattagaaacagatcctgctgctccaccacctgcagcgtgttcaaaacaacaagattcaatgaggttctgccctctgccctcagctcacgtctcagcgtcagctgcaaaaagtcactcaagtcctcagccactacagctgacaattcagagccagggctaagcgtgggactggcaagcgtgagtgagtaccacccaaaaactgcatgctggaataagctctctttgtgtcaccggtgatgccttccaataggtgagtgataaagcgaggtagtttttctttaatcatttgagtaatagaaaagtcctctaaataagtcactaggaccccaggaaccacaatgtggtagctgacagcgtgtcgctcaagcatggttagtagagatgagagtctgaaaaacagaaagcatgcactaaaccagagttgccagtctcactgaaggaaaaatcactctctccagcagcaaagtgcccactgggtggccctctcggacatacaaaaatcgatccgtgtggttaaagagcagcacagttagctcctgtcttctcccagcaaagatcacatcggactgggttagtatgcccctggaatggtagtcattcaaggccataaatctgccttggtagccattaggaatcagcacgctcactctcaagtgaaccaaaaccaccccatgcggaggaatgtggaaagattctgggcaaaaaaaggtatatctattgctagtcccttcctggacgggagcaatccctccagggctatctatgaaagcatacagagattcagccatagctcagcccgcttaccagtagacagagagcacagcagtacaagcgccaacagcagcgactgactacccactgacccagctccctatttaaaggcaccttacactgacgtaatgaccaaaggtctaaaaaccccgccaaaaaaacacacacgccctgggtgtttttcgcgaaaacacttccgcgttctcacttcctcgtatcgatttcgtgactcaacttccgggttcccacgttacgtcacttctgcccttacatgtaactcagccgtagggcgccatcttgcccacgtccaaaatggcttccatgtccggccacgcctccgcggcgaccgttagccgtgcgtcgtgacgtcatttgcatcaccgtttctcgtccaatcagcgttggctccgccccaaaaccgttaaaattcaaaagctcatttgcatattaacttttgtttactttgtggggtatattattgatgatg

SEQ ID NO: 6 - DNA, 34808 bp, Artificial Sequence
  Synthetic Parental sequence of recombinant simian adenovirus serotype 25, wherein the E1 and E3 regions are deleted
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatacatggcccgaaaggagcgatgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 7 - DNA, 34742 bp, Artificial Sequence
  Synthetic Parental sequence of recombinant human adenovirus serotype 5, wherein the E1 and E3 regions are deleted
ccatcttcaataatatacctcaaacttttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttgcgatgcatgcgcgttgtcaaatatgagctcacaatgcttccatcaaacgagttggtgctcatggcggcggcggctgctgcaaaacagatacaaaactacataagacccccaccttatatattctttcccacccttaaccacgcccagatccgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagactcgagcggccgcgatatcttatgtgtaatgtaatttgactcctttgagcacgggctcagaatcgtcctcgtcgaacttgcagcagctgccacagctacaacagcccttcaggcagctacagcagctggtcatgcaacacagcatgattgtgaccatcacgatggcaatcagtccggcgataaagcccagccagatgtaccagggccacttgatgtactgctcgtacttccccagttcttgcaggtcgatcaggctctcgttcagattcttggccacctcgttcagccggtcgatctctttctggatgttcacgacgctggcattgattccgctgatgtcgcccaggtccacgtcggggcttgtgtggttcttaaagtacttatccagttcctctttgaagctgtccagctcgggctgcagagggtcgtacacggtattgttcacaatgccgatcacgacgtcgcagttgccagacacgaaggtgttgtcggtggtgatgatctggggctcgtagaagttccgctgggtcacgaaccaatgggtgccgttggacacgaacacgccttctctaggaaagtgggctttgccgtcgtggcagatggctggagcggtggtgaaattcttctcttgagcgggcacgtatgtcacgtgcagaaacaccacgccgtgaggggcagactgagggaagctcatcaggtggtagcccttgccgcaaaagtccactctcttgctctggcccagcacacactcagacatcttggtggcggccagattggcagaggctctaatctcggcggctctgatcagctgctgggtaacgtaggtctgcagggactgcagccttccggtgatcagtctgtcgatctgcacctcggcttccaccttgtccagtctgctcaggatgtcgttcagcacagagctgatggcgccgaagttggaggacagctgcttgaccagggtgttcagtgcctgggcattctggttgaccacgtcctgcagctttcccagggcgcttgctgtgctgctcaggctgtcctggatcttgccgatggcgctgttgaactggttggcgatcagcttctggttctcgtacagcacattctgggtcactccgatgccgttgaaccggtaggccatctgcatagcaaaggggatctgcagagcggcgccagctccaaatgtccagccgcttgtgattgtgccggccagcagggcagatgtgtactgggcgatcatctcatcggtcagcagtggtggcagcactgtcagtccgttaaacttctgggcgcaaatcagatccctggcggcaatgtcgcccagacaatcgccatactgcttgatgaagccggcgtcggccagtgtcactttgttgaacagcaggtcctcgatgaagctccgcttgctgggcttgctaggatcgggcagaatctggctgaaattgaagccgccgaagtccttgataggaggggtcttgtagatctgcttcacttgggcgaacacctcttgggtgttcttgtcctgttccacggcgatccctgtcagggctctattcagctgggtgcagaagctgccgtactgcagcagcaggttggagcactcggtggaatcgccgcagatgtacatggtgcagtccacgctggtcttggtcatggacacaggcaggatctctgtggtcacgctgatggtgaagttggtggggatagcgatagagttgttggagtaggccacgctgttctcggcgcccagagacattgtgtaggcaatgatgctctggctggccacagatctggcccgtctggggctgtttgtctgtgtctggtagctggcacagatgccagcgccgatggggatgtcgcactcgtagctattgttcacgtgctcggctccgatcagacagccggctctggtctgaaacacattgctgccggtggagtacacccgccatgtaggtgtcagctgatcggcgtgaatggccacgggcacttcggtacagttcacgtcctggtacagcactgccacctgattgctggtgttggtgccaggggtgatcacagacactccgccgaagctgcaaggggtgatgtccaggatttccagtgtctggggatctcgtacggcgtctgtggtatcggcaatatcccggccaaactgctggaatggcaggaacttcttgttgctctctgtcagcacgccggtgccggtcaggccgttgaagttgaagttcacgcatttgttcttcacgagattggtgcttttcttagggccgcacactgtggcaggggcatgcagcagttcgaagctcagcaccaccactctgtagggctgatagcccacgccatttgtgggctgaaagccgtaggactgcagtgggaagtagcagttgaagccttccacgccgttacaaggggtgctgccggcctgatagatctcggtggagatgtcccgctcgaagggcttcagattggacttccggaacagccggtacaggtaattgtagttgccgccgactttggagtccaggttgttgctgttccaggcaatcacacagccggtgaagtcgtcgggcagcttgtagttgtagtcggcgatcttgcctgtctgtccaggggcaatctgccgcacttcatctccccggatcacgaagctgtcggcgtacacgtttgtgaagcacaggtcgttcagcttggtaggggacacgccgtagcacttgaaggtgctgaagctggcggagttgtacagcacggagtagtcggccacgcaattgctgatccgcttccggttccaggcgtacacagaggcgaatctggtggcattgaacacctcgccgaaggggcacagattggtgatattggggaaccgcacgatggattcggtgggctgcacccggaagttgctggtctggtagatgcccttttccacggtgaaggacttcagggtgcactttgtctcgctcagaggatccagagcacaatccacggcgtcggtgatggtgccgttctcgttgtacttcagcaggaaggttctaggctgcaggtagcccacatagtaagcggcggcaccagctgtccatccgctgctgctatcgccaggtgtcaggtagcttctgtgcagggccagcagtgtctgaaaccgggtgatgttgatgccgatgggcagatccaccaggggttccagagcagagaagccctgaggcagatcccgcacgaggttgataggggtgtgcttgctgtagatcttgaagtagccgtcgatgttcttgaacacgaactcgcgcaggttcttgaagttgccctgcttgccttccaggtccatcaggaaaggctgggacacgtactcgaaggtgcagttgttggcgctgctgtacacccggaactcgctttccatccagctcttgttgttcttgtgatagtagacgcccaggaaggggtcgttgcagaactggaactcgcacactttgatgaccacgttggtggcgttgttcacgatcagcaggctctgggtcttgctgtccagtgtggtgccgaagatccagcctctgatgatgttggacttctcggtgctggcaaagtacaccccgtcgttgaagggcagcacggggttgtcgaatctcttggtgccattggtgccggacacgtggatggcgtggaaccaggtcacgttgctgaagaaaggcaggaacaggtcctgggtagagtgcagcacgctggatctgaacaccttgtcggggtagtacacgcctctggtaaagctgttggtgtaggctggaggcagctgggttcttgtggtcaggttcacacactgactagagactagtggcaataacacaaggaacacaaacatggtaccagatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattacagtattacgcgctatgagtaacacaaaattattcagatttcacttcctcttattcagttttcccgcgaaaatggccaaatcttactcggttacgcccaaatttactacaacatccgcctaaaaccgcgcgaaaattgtcacttcctgtgtacaccggcgcgctgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatgtcgacccccggtcccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccactacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggatgcggtggccatccaccagtgcaagagaggcatcttctgcctggtgaaacaggccaagatctcctacgaggtcactccaaacgaccatcgcctctcctacgagctcctgcagcagcgccagaagttcacctgcctggtcggagtcaaccccatcgtcatcacccagcagtctggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgggggagggaagaacaggaagaaccatgattaacttttaatccaaacggtctcggagtacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcgtgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtggacgtttagagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtaaataaatctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg